(19) United States
(12) Patent Application Publication
(10) Pub. No.: US 2024/0009195 A1
(43) Pub. Date: Jan. 11, 2024

(54) NOVEL USES

(71) Applicant: INTRA-CELLULAR THERAPIES, INC., New York, NY (US)

(72) Inventors: Peng LI, New Milford, NJ (US); David KASS, New York, NY (US); Robert DAVIS, San Diego, CA (US); Gretchen SNYDER, New York, NY (US)

(21) Appl. No.: 18/007,018

(22) PCT Filed: Jul. 26, 2021
(86) PCT No.: PCT/US2021/043230

§ 371 Date: Jan. 26, 2023

Related U.S. Application Data
(60) Provisional application No. 63/056,706, filed on Jul. 26, 2020.

(51) Int. Cl. A61K 31/519; A61K 31/7034; A61K 45/06; A61P 9/04

(52) U.S. Cl. CPC A61K 31/519; A61K 31/7034; A61K 45/06; A61P 9/04

(57) ABSTRACT

The disclosure provides methods, treatments and materials for treating diseases or disorders mediated by cyclic nucleotides and/or sodium glucose cotransporter 2. In particular, the present disclosure provides for methods of treating such diseases and disorders with a PDE1 inhibitor in combination with additional therapeutic agents.

### CROSS-REFERENCE TO RELATED APPLICATIONS

This application is an international application which claims priority to and the benefit of U.S. Provisional Application No. 63/056,706, filed on Jul. 26, 2020, the contents of which are hereby incorporated by reference in its entirety.

### FIELD OF THE DISCLOSURE

The field of the present disclosure relates to methods, treatments and materials for treating diseases or disorders related to decreased circulating levels of glucagon and subsequent loss of direct and indirect beneficial effects on end organs including the heart and kidneys. This disclosure provides for methods of treating such diseases and disorders with a PDE1 inhibitor to slow cyclic nucleotide hydrolysis in combination with therapeutic agents that modulate circulating levels of glucagon or modulate glucagon receptor activity and directly or indirectly increase intracellular levels of cAMP.

### BACKGROUND

Eleven families of phosphodiesterases (PDEs) have been identified but only PDEs in Family I, the Ca2+/calmodulin-dependent phosphodiesterases (CaM-PDEs), which are activated by Ca2+/calmodulin and have been shown to mediate the calcium/calmodulin cyclic nucleotide (e.g. cGMP and cAMP) dependent signaling pathways. The three known CaM-PDE genes, PDE1A, PDE1B, and PDE1C, are all expressed in central nervous system tissue. PDE1A is expressed in the brain, lung, kidney and heart. PDE1B is primarily expressed in the central nervous system, but it is also detected in monocytes and neutrophils and has been shown to be involved in inflammatory responses of these cells. PDE1C is expressed in olfactory epithelium, cerebellar granule cells, striatum, kidney, heart, and vascular smooth muscle. PDE1C has been demonstrated to be a major regulator of proliferation and function in human smooth muscle.

Cyclic nucleotide phosphodiesterases down-regulate intracellular cAMP and cGMP signaling by hydrolyzing these cyclic nucleotides to their respective 5′-monophosphates (5′AMP and 5′GMP), which are inactive in terms of intra-cellular signaling pathways. Both cAMP and cGMP are central intracellular second-messengers and they play roles in regulating numerous cellular functions. PDE1A and PDE1B preferentially hydrolyze cGMP over cAMP, while PDE1C shows approximately equal cGMP and cAMP hydrolysis.

A major component of cardiac dysfunction in heart failure (HF) resides in second messenger signaling defects coupled to cyclic 3′, 5′-cyclic adenosine and guanosine monophosphate (cAMP, cGMP) that limit functional reserve. Cyclic AMP stimulates protein kinase A (PKA) and exchange protein activated by cAMP (EPAC), acutely enhancing excitation-contraction coupling and sarcomere function. Cyclic GMP acts as a brake on this signaling by activating protein kinase G. Both cyclic nucleotides have relevant vascular and fibroblast activity, reducing vessel tone, altering permeability and proliferation, and suppressing fibrosis. Degradation (hydrolysis) of these cyclic nucleotides is accomplished by cyclic nucleotide phosphodiesterases (PDEs). PDE1 activity is believed to be altered in chronic disease conditions such as diabetes mellitus, atherosclerosis, cardiac pressure-load stress and heart failure, as well as in response to long-term exposure to nitrates.

In cardiac fibroblasts, PDE1A is highly upregulated after stimulation with ATII and TGFβ. Moreover, PDE1 inhibitors have been reported to decrease ATII or TGFβ induced cardiac myofibroblast activation, ECM production, and profibrotic gene expression, suggesting that PDE1 inhibition also mediates the antifibrotic effects via cAMP. The PDE1 isozymes are also abundant in the kidney. Thus, it follows that increased cAMP levels induced by specific PDE1 inhibitors could be beneficial in treating renal diseases.

For example, kidney fibrosis is an important factor for the progression of kidney diseases, such as diabetes mellitus induced kidney failure, glomerulosclerosis and nephritis resulting in chronic kidney disease or end-stage renal disease. Cyclic adenosine monophosphate (cAMP) and cyclic guanosine monophosphate (cGMP) have been implicated to suppress several known renal diseases through a number of complex mechanisms, such as the nitric oxide/ANP/guanylyl cyclases/cGMP-dependent protein kinase and cAMP/Epac/adenylyl cyclases/cAMP-dependent protein kinase pathways. From these diverse mechanisms it has been proposed that new pharmacological treatments will evolve for the therapy or even prevention of kidney failure.

A robust association exists between diabetes mellitus (DM) and certain diseases including heart failure and kidney disease. Increased levels of glucagon in DM have been considered deleterious because of alterations in its counterregulatory role to insulin. However, glucagon's physiological role is broad and glucagon plays an important role in the maintenance of organs expressing glucagon receptor as both in heart and kidney. For example, glucagon has been shown to be therapeutically useful in treating heart failure and to increase cardiac index and cardiac output while decreasing vascular resistance. These effects are thought to be mediated by glucagon receptor activation and subsequent increases in intracellular cyclic AMP signaling in the heart. In the kidneys, glucagon induces vasodilation, and increases renal plasma flow, glomerular filtration rate and electrolyte excretion.

Certain treatments for DM, such as sodium glucose co-transporter 2 (SGLT2) inhibitors, are known to elevate glucagon levels and have been shown to have beneficial effects in treating heart and kidney failure in humans with and without DM. Conversely, other DM treatments, for example the dipeptidyl peptidase-4 inhibitors, decrease glucagon and have raised concerns that their use may precipitate heart failure. Therefore, it is important to isolate glucagon's hemodynamic action from its effects on glucose control. For example, the hemodynamic actions of SGLT2 inhibitors have been proposed to be independent of effects on glucose control and may act indirectly on the heart via increased glucagon secretion to alter myocardial metabolism, ion transporters, fibrosis, adipokines, and vascular function. These actions also may be beneficial in the preservation of renal function as well.

There is a need for additional treatment choices for patients suffering from metabolic, cardiac and renal diseases. New, safer and selective strategies for treating such disorders are needed.

### SUMMARY

Provided herein are methods for treating diseases or disorders associated with altered glucagon function by the administration of at least one PDE1 inhibitor to enhance and maintain glucagon receptor mediated increases in intracellular cAMP and at least one agent that modulates circulating glucagon levels or modulates glucagon receptor activity (e.g., glucagon, SGLT2 inhibitors or other agents that may directly or indirectly increase circulating glucagon in plasma).

Recently, the inventors have found that, in mammals where PDE1C is the predominant PDE1 isoform in cardiac tissue, PDE1 inhibition has acute positive inotropic, lusitropic, and arterial vasodilatory effects via the ability of these inhibitors to enhance cyclic nucleotide signaling in the cardiovascular system. Recent studies have also shown that the cyclic nucleotides, cAMP and cGMP, play a prominent role in progressing renal disorders, such as, kidney fibrosis, chronic kidney disease, kidney fibrosis, renal failure, glomerulosclerosis and nephritis.

Glucagon has historically been administered to treat heart failure. Glucagon administration can exert a positive action on cardiovascular performance by increasing cardiac index (cardiac output and contractility), and/or by decreasing peripheral vascular resistance. SGLT2 inhibitors also are known to elevate circulating levels of glucagon and have been found to exert an important role in the maintenance of both heart and kidney function.

Without being bound by theory, it is believed that by modulating the circulating levels of glucagon, PDE1 inhibitors can be used in combination with glucagon or other agents that increase glucagon function to reduce the effective dose of the PDE1 inhibitor and/or the effective dose of the glucagon or glucagon modulating agent, for example a SGLT2 inhibitor, or to reduce the undesirable side effects of glucagon or glucagon modulating agent (e.g., mycotic infections, urinary tract infections, osmotic diuresis, and diabetic ketoacidosis).

Thus, in a first aspect, the present disclosure provides a method for the treatment or prophylaxis of a disease, disorder or condition mediated by altered glucagon function, comprising administration of a pharmaceutically effective amount of a PDE1 inhibitor (e.g., a PDE1 inhibitor of Formula I, Ia, II, III, IV, V, VI and/or VII as herein described) and a pharmaceutically effective amount of a second active agent that increases circulating glucagon (e.g., SGLT2 inhibitor and/or glucagon) to a patient in need thereof. For example, the disease or condition mediated by altered glucagon function may be diabetes mellitus (e.g., type 2 diabetes mellitus), kidney fibrosis, chronic kidney disease, kidney fibrosis, renal failure, glomerulosclerosis, nephritis, heart failure (e.g., chronic heart failure, acute heart failure or heart failure consequent to myocardial infarction), angina, stroke, renal failure, essential hypertension, pulmonary hypertension, secondary hypertension, isolated systolic hypertension, hypertension associated with diabetes, hypertension associated with atherosclerosis, renovascular hypertension, congestive heart failure, an inflammatory disease or disorder, fibrosis, cardiac hypertrophy, vascular remodeling, a connective tissue disease or disorder (e.g., Marfan Syndrome), chronic heart failure, myocardial ischemia, myocardial hypoxia, reperfusion injury, left ventricular dysfunctions (e.g., myocardial infarction, ventricular expansion), vascular leakage (i.e., consequent to hypoxia), muscular dystrophy (e.g., Duchenne muscular dystrophy), or amyotrophic lateral sclerosis.

In a second aspect, the present disclosure provides for a combination therapy comprising a PDE1 inhibitor (e.g., a compound according to any of Formulas I, Ia, II, III, IV, V, VI and/or VII) and a second active agent that increases circulating glucagon (e.g., SGLT2 inhibitor and/or glucagon).

### BRIEF DESCRIPTION OF THE FIGURES

FIG. 1 illustrates the average change in cardiomyocyte sarcomere shortening upon co-administration of glucagon with a PDE1 inhibitor according to the present disclosure.

FIG. 2 illustrates the peak intracellular Ca2+ transient amplitude in cardiomyocytes upon co-administration of glucagon with a PDE1 inhibitor according to the present disclosure.

### DETAILED DESCRIPTION OF THE DISCLOSURE

In some embodiments, the PDE1 inhibitors for use in the methods of treatment and prophylaxis described herein are selective PDE1 inhibitors.

### Compounds of the Disclosure

In one embodiment the present disclosure provides that the PDE1 inhibitors for use in the methods of treatment and prophylaxis described herein are compounds of Formula I.

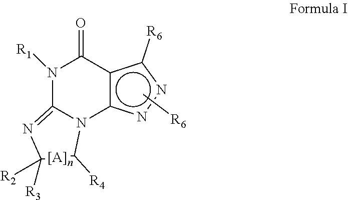

wherein
(i) R₁ is H or C₁₋₄ alkyl (e.g., methyl);
(ii) R₄ is H or C₁₋₄ alkyl and R₂ and R₃ are, independently, H or C₁₋₄ alkyl (e.g., R₂ and R₃ are both methyl, or R₂ is H and R₃ is isopropyl), aryl, heteroaryl, (optionally hetero) arylalkoxy, or (optionally hetero)arylalkyl; or R₂ is H and R₃ and R₄ together form a di-, tri- or tetramethylene bridge (pref. wherein the R₃ and R₄ together have the cis configuration, e.g., where the carbons carrying R₃ and R₄ have the R and S configurations, respectively);
(iii) R₅ is a substituted heteroarylalkyl, e.g., substituted with haloalkyl; or R₅ is attached to one of the nitrogens on the pyrazolo portion of Formula I and is a moiety of Formula A

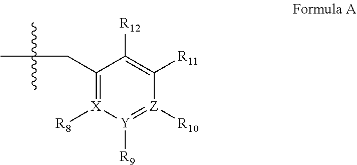

wherein X, Y and Z are, independently, N or C, and R₈, R₉, R₁₁ and R₁₂ are independently H or halogen (e.g., Cl or F), and Rio is halogen, alkyl, cycloalkyl, haloalkyl (e.g., trifluoromethyl), aryl (e.g., phenyl), heteroaryl (e.g., pyridyl (for example pyrid-2-yl) optionally substituted with halogen, or thiadiazolyl (e.g., 1,2,3-thiadiazol-4-yl)), diazolyl, triazolyl, tetrazolyl, arylcarbonyl (e.g., benzoyl), alkylsulfonyl (e.g., methylsulfonyl), heteroarylcarbonyl, or alkoxycarbonyl; provided that when X, Y, or Z is nitrogen, R₈, R₉, or R₁₀, respectively, is not present; and
(iv) R₆ is H, alkyl, aryl, heteroaryl, arylalkyl (e.g., benzyl), arylamino (e.g., phenylamino), heteroarylamino, N,N-dialkylamino, N,N-diarylamino, or N-aryl-N-(arylalkyl)amino (e.g., N-phenyl-N-(1,1′-biphen-4-ylmethyl)amino); and
(v) n=0 or 1;
(vi) when n=1, A is —C(R₁₃R₁₄)—
   wherein R₁₃ and R₁₄, are, independently, H or C₁₋₄ alkyl, aryl, heteroaryl, (optionally hetero) arylalkoxy or (optionally hetero)arylalkyl;
      in free, pharmaceutically acceptable salt or prodrug form, including its enantiomers, diastereoisomers and racemates.

In another embodiment the present disclosure provides that the PDE1 inhibitors for use in the methods as described herein are Formula 1a:

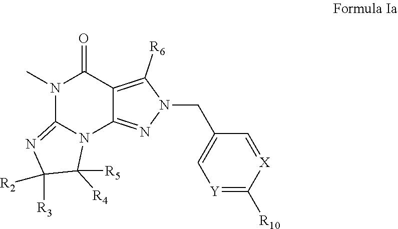

wherein
(i) R₂ and R₅ are independently H or hydroxy and R₃ and R₄ together form a tri- or tetra-methylene bridge [pref. with the carbons carrying R₃ and R₄ having the R and S configuration respectively]; or R₂ and R₃ are each methyl and R₄ and R₅ are each H; or R₂, R₄ and R₅ are H and R₃ is isopropyl [pref the carbon carrying R₃ having the R configuration];
(ii) R₆ is (optionally halo-substituted) phenylamino, (optionally halo-substituted) benzylamino, C₁₋₄alkyl, or C₁₋₄alkyl sulfide; for example, phenylamino or 4-fluorophenylamino;
(iii) R₁₀ is C₁₋₄alkyl, methylcarbonyl, hydroxyethyl, carboxylic acid, sulfonamide, (optionally halo- or hydroxy-substituted) phenyl, (optionally halo- or hydroxy-substituted) pyridyl (for example 6-fluoropyrid-2-yl), or thiadiazolyl (e.g., 1,2,3-thiadiazol-4-yl); and
X and Y are independently C or N,
in free, pharmaceutically acceptable salt or prodrug form, including its enantiomers, diastereoisomers and racemates.

In another embodiment the present disclosure provides that the PDE1 inhibitors for use in the methods of treatment and prophylaxis described herein are compounds of Formula II:

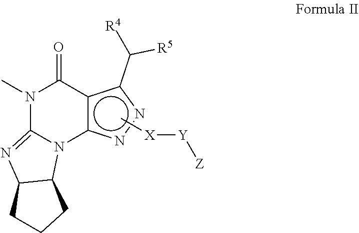

(i) X is C₁₋₆alkylene (e.g., methylene, ethylene or prop-2-yn-1-ylene);
(ii) Y is a single bond, alkynylene (e.g., —C≡C—), arylene (e.g., phenylene) or heteroarylene (e.g., pyridylene);
(iii) Z is H, aryl (e.g., phenyl), heteroaryl (e.g., pyridyl, e.g., pyrid-2-yl), halo (e.g., F, Br, Cl), haloC₁₋₆alkyl (e.g., trifluoromethyl), —C(O)—R₁, N(R²)(R³), or C₃₋₇cycloalkyl optionally containing at least one atom selected from a group consisting of N or O (e.g., cyclopentyl, cyclohexyl, tetrahydro-2H-pyran-4-yl, or morpholinyl);
(iv) R¹ is C₁₋₆alkyl, haloC₁₋₆alkyl, OH or —OC₁₋₆alkyl (e.g., —OCH₃);
(v) R² and R³ are independently H or C₁₋₆alkyl;
(vi) R⁴ and R⁵ are independently H, C₁₋₆alky or aryl (e.g., phenyl) optionally substituted with one or more halo (e.g., fluorophenyl, e.g., 4-fluorophenyl), hydroxy (e.g., hydroxyphenyl, e.g., 4-hydroxyphenyl or 2-hydroxyphenyl) or C₁₋₆alkoxy;
(vii) wherein X, Y and Z are independently and optionally substituted with one or more halo (e.g., F, Cl or Br), C₁₋₆alkyl (e.g., methyl), haloC₁₋₆alkyl (e.g., trifluoromethyl), for example, Z is heteroaryl, e.g., pyridyl substituted with one or more halo (e.g., 6-fluoropyrid-2-yl, 5-fluoropyrid-2-yl, 6-fluoropyrid-2-yl, 3-fluoropyrid-2-yl, 4-fluoropyrid-2-yl, 4,6-dichloropyrid-2-yl), haloC₁₋₆alkyl (e.g., 5-trifluoromethylpyrid-2-yl) or C₁₋₆-alkyl (e.g., 5-methylpyrid-2-yl), or Z is aryl, e.g., phenyl, substituted with one or more halo (e.g., 4-fluorophenyl), in free, pharmaceutically acceptable salt or prodrug form, including its enantiomers, diastereoisomers and racemates.

In yet another embodiment the present disclosure provides that the PDE1 inhibitors for use in the methods of treatment and prophylaxis described herein are Formula III:

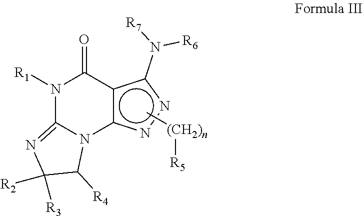

wherein
(i) R₁ is H or C₁₋₄ alkyl (e.g., methyl or ethyl);
(ii) R₂ and R₃ are independently H or C₁₋₆ alkyl (e.g., methyl or ethyl); (iii) R₄ is H or C₁₋₄ alkyl (e.g., methyl or ethyl);
(iv) R₅ is aryl (e.g., phenyl) optionally substituted with one or more groups independently selected from —C(═O)—C₁₋₆ alkyl (e.g., —C(═O)—CH₃) and C₁₋₆-hydroxyalkyl (e.g., 1-hydroxyethyl);
(v) R₆ and R₇ are independently H or aryl (e.g., phenyl) optionally substituted with one or more groups independently selected from C₁₋₆ alkyl (e.g., methyl or ethyl) and halogen (e.g., F or Cl), for example unsubstituted phenyl or phenyl substituted with one or more halogen (e.g., F) or phenyl substituted with one or more C₁₋₆ alkyl and one or more halogen or phenyl substituted with one C₁₋₆ alkyl and one halogen, for example 4-fluorophenyl or 3,4-difluorophenyl or 4-fluoro-3-methylphenyl; and
(vi) n is 1, 2, 3, or 4,
   in free, pharmaceutically acceptable salt or prodrug form, including its enantiomers, diastereoisomers and racemates.

In yet another embodiment the present disclosure provides that the PDE1 inhibitors for use in the methods of treatment and prophylaxis described herein are Formula IV

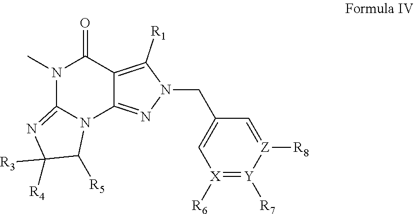

wherein
(i) R₁ is C₁₋₄alkyl (e.g., methyl or ethyl), or —NH(R₂), wherein R₂ is phenyl optionally substituted with halo (e.g., fluoro), for example, 4-fluorophenyl;
(ii) X, Y and Z are, independently, N or C;
(iii) R₃, R₄ and R₅ are independently H or C₁₋₄alkyl (e.g., methyl); or R₃ is H and R₄ and R₅ together form a tri-methylene bridge (pref wherein the R₄ and R₅ together have the cis configuration, e.g., where the carbons carrying R₄ and R₅ have the R and S configurations, respectively),
(iv) R₆, R₇ and R₅ are independently:
   H,
   C₁₋₄alkyl (e.g., methyl),
   pyrid-2-yl substituted with hydroxy, or
   —S(O)₂—NH₂;
provided that when X, Y and/or Z are N, then R₆, R₇ and/or R₈, respectively, are not present; and when X, Y and Z are all C, then at least one of R₆, R₇ or R₈ is —S(O)₂—NH₂ or pyrid-2-yl substituted with hydroxy,
in free, pharmaceutically acceptable salt or prodrug form, including its enantiomers, diastereoisomers and racemates.

In another embodiment the present disclosure provides that the PDE1 inhibitors for use in the methods as described herein are Formula V:

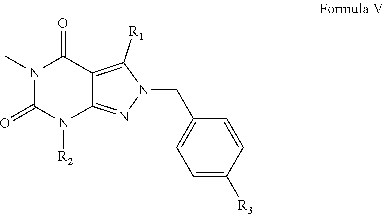

wherein
(i) R₁ is —NH(R₄), wherein R₄ is phenyl optionally substituted with halo (e.g., fluoro), for example, 4-fluorophenyl;
(ii) R₂ is H or C₁₋₆alkyl (e.g., methyl, isobutyl or neopentyl);
(iii) R₃ is —SO₂NH₂ or —COOH;
in free, pharmaceutically acceptable salt or prodrug form, including its enantiomers, diastereoisomers and racemates.

In another embodiment the present disclosure provides that the PDE1 inhibitors for use in the methods as described herein are Formula VI:

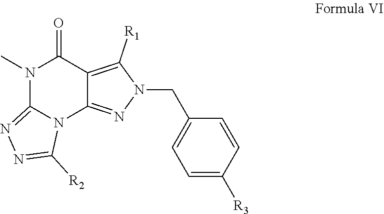

wherein
(i) R₁ is —NH(R₄), wherein R₄ is phenyl optionally substituted with halo (e.g., fluoro), for example, 4-fluorophenyl;
(ii) R₂ is H or C₁₋₆alkyl (e.g., methyl or ethyl);
(iii) R₃ is H, halogen (e.g., bromo), C₁₋₆alkyl (e.g., methyl), aryl optionally substituted with halogen (e.g., 4-fluorophenyl), heteroaryl optionally substituted with halogen (e.g., 6-fluoropyrid-2-yl or pyrid-2-yl), or acyl (e.g., acetyl),
in free, pharmaceutically acceptable salt or prodrug form, including its enantiomers, diastereoisomers and racemates.

In another embodiment the present disclosure provides that the PDE1 inhibitors for use in the methods as described herein are Formula VII:

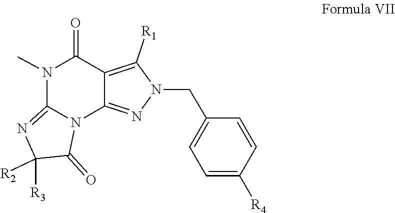

(i) R₁ is —NH(R₅), wherein R₅ is phenyl optionally substituted with halo (e.g., fluoro), for example, 4-fluorophenyl;
(ii) R₂ and R₃ are each independently H or C₁₋₆alkyl (e.g., methyl or ethyl);
(iii) R₄ is aryl optionally substituted with halogen (e.g., 4-fluorophenyl) or heteroaryl optionally substituted with halogen (e.g., 6-fluoropyrid-2-yl),
in free, pharmaceutically acceptable salt or prodrug form, including its enantiomers, diastereoisomers and racemates.

In one embodiment, the present disclosure provides for administration of a PDE1 inhibitor for use in the methods described herein (e.g., a compound according to Formulas I, Ia, II, III, IV, V, VI and/or VII), wherein the inhibitor is a compound according to the following:

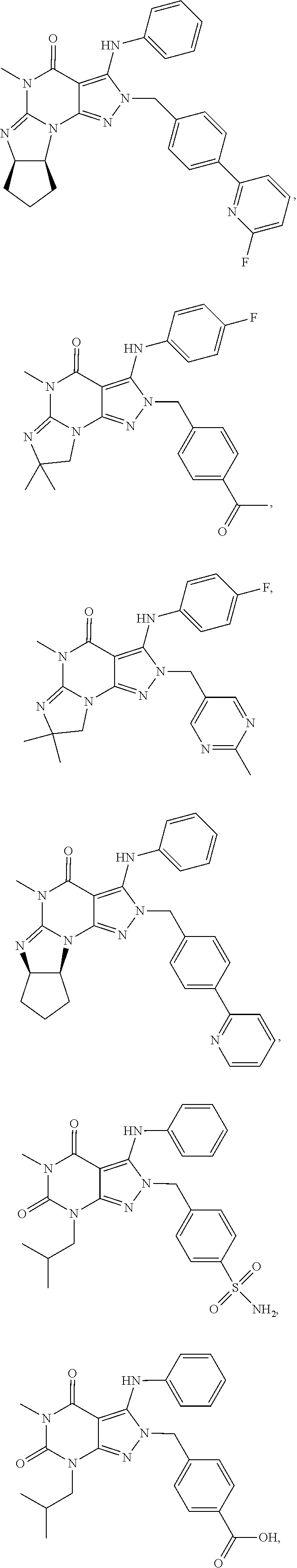

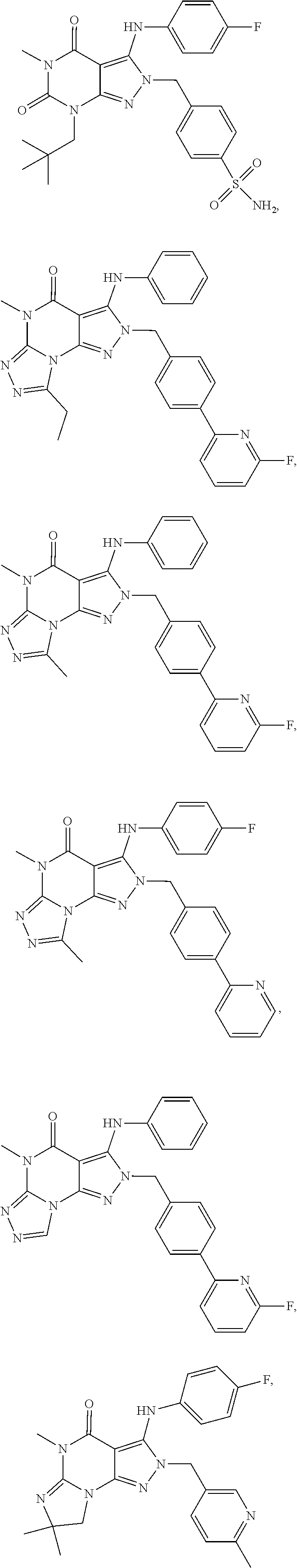

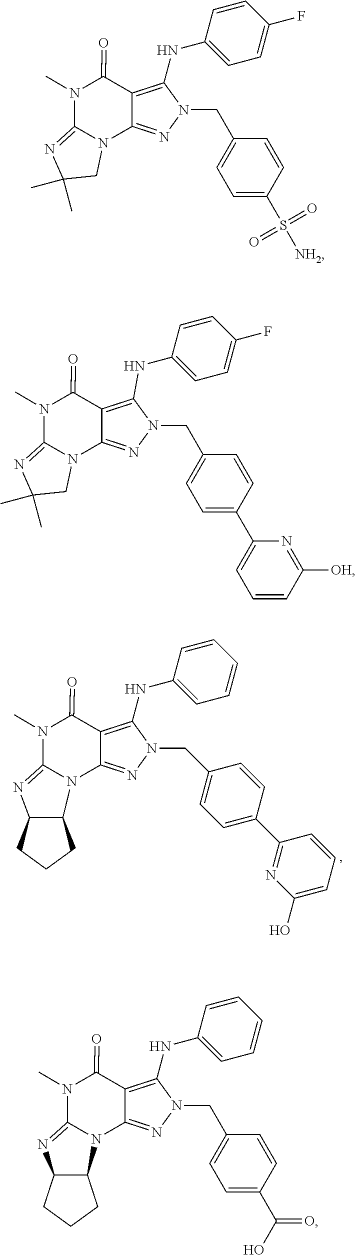 [SMILES: CN1C(=O)c2c(nn(Cc3ccc(S(N)(=O)=O)cc3)c2Nc2ccc(F)cc2)N2CC(C)(C)N=C12 ; CN1C(=O)c2c(nn(Cc3ccc(-c4cccc(O)n4)cc3)c2Nc2ccc(F)cc2)N2CC(C)(C)N=C12 ; CN1C(=O)c2c(nn(Cc3ccc(-c4cccc(O)n4)cc3)c2Nc2ccccc2)N2C1=N[C@@H]1CCC[C@@H]12 ; CN1C(=O)c2c(nn(Cc3ccc(C(=O)O)cc3)c2Nc2ccccc2)N2C1=N[C@@H]1CCC[C@@H]12]

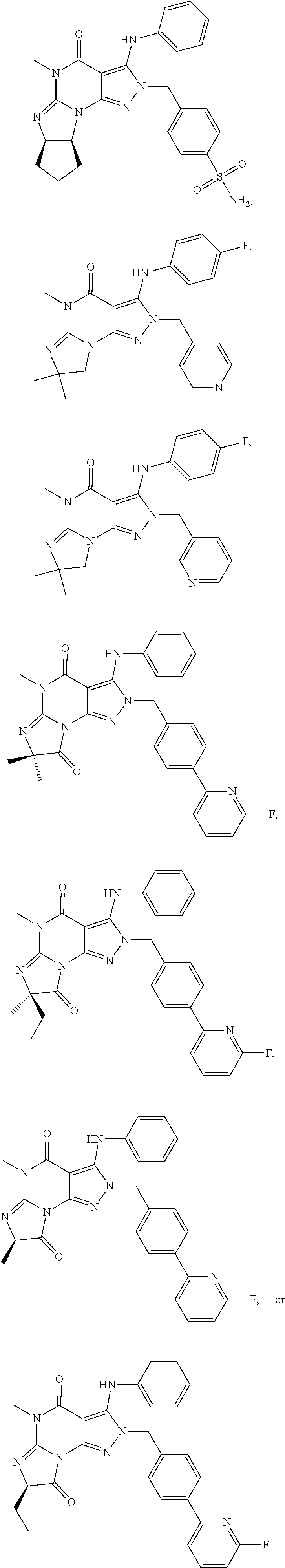

In one embodiment the present disclosure provides administration of a PDE1 inhibitor for use in the methods described herein, wherein the inhibitor is a compound according to the following:

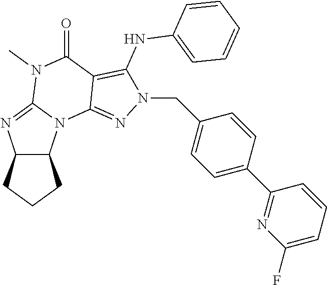 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.

In another embodiment, the present disclosure provides administration of a PDE1 inhibitor for use in the methods described herein, wherein the inhibitor is a compound according to the following:

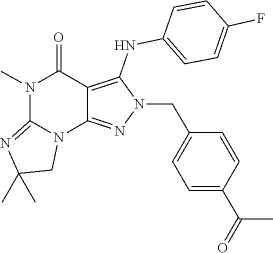 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.

In still another embodiment, the present disclosure provides administration of a PDE1 inhibitor for use in the methods described herein, wherein the inhibitor is a compound according to the following:

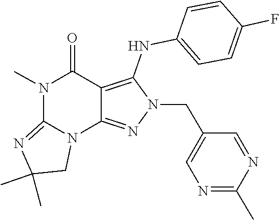 [SMILES: Cc1ncc(Cn2nc3c(c2Nc2ccc(F)cc2)C(=O)N(C)C2=NC(C)(C)CN23)cn1]

in free or pharmaceutically acceptable salt form.

In still another embodiment, the present disclosure provides administration of a PDE1 inhibitor for use in the methods described herein, wherein the inhibitor is a compound according to the following:

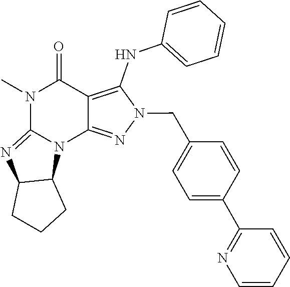 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.

In still another embodiment, the present disclosure provides administration of a PDE1 inhibitor for use in the methods described herein, wherein the inhibitor is a compound according to the following.

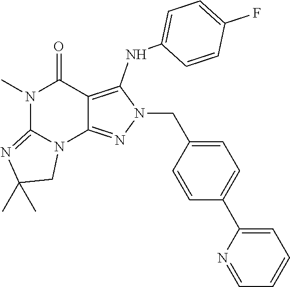 [SMILES: CN1C(=O)c2c(nn(Cc3ccc(-c4ccccn4)cc3)c2Nc2ccc(F)cc2)N2CC(C)(C)N=C12]

in free or pharmaceutically acceptable salt form.

In one embodiment, selective PDE1 inhibitors of any of the preceding formulae (e.g., Formulas I, Ia, II, III, IV, V, VI and/or VII) are compounds that inhibit phosphodiesterase-mediated (e.g., PDE1-mediated, especially PDE1B-mediated) hydrolysis of cGMP, e.g., the preferred compounds have an IC50 of less than 1 μM, preferably less than 500 nM, preferably less than 50 nM, and preferably less than 5 nM in an immobilized-metal affinity particle reagent PDE assay, in free or salt form.

In other embodiments, the present disclosure provides administration of a PDE1 inhibitor for treatment according to the methods described herein, wherein the inhibitor is a compound according to the following:

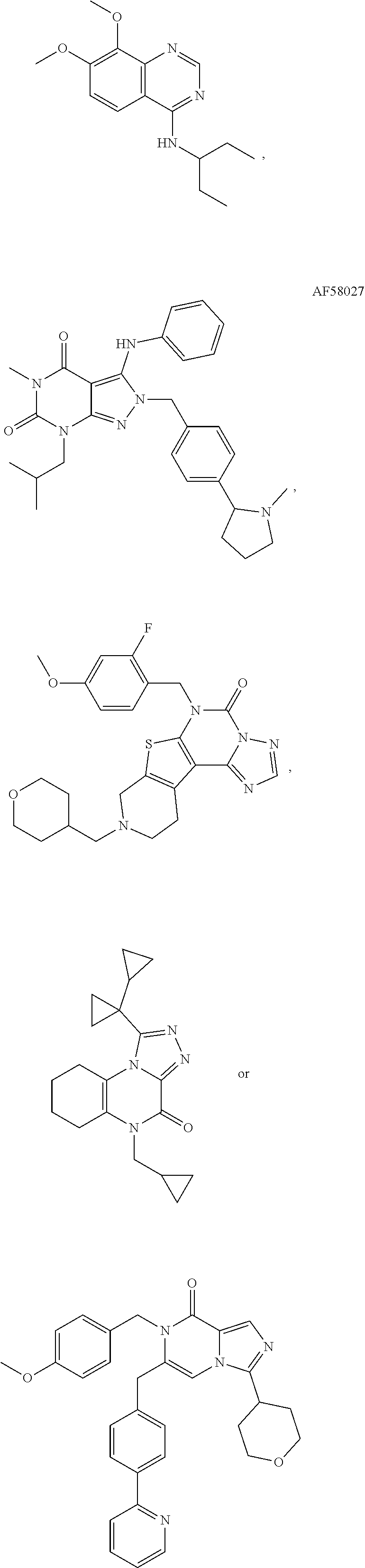

Further examples of PDE1 inhibitors suitable for use in the methods and treatments discussed herein can be found in International Publication WO2006133261A2; U.S. Pat. Nos. 8,273,750; 9,000,001; 9,624,230; International Publication WO2009075784A1; U.S. Pat. Nos. 8,273,751; 8,829,008; 9,403,836; International Publication WO2014151409A1, U.S. Pat. Nos. 9,073,936; 9,598,426; 9,556,186; U.S. Publication 2017/0231994A1, International Publication WO2016022893A1, and U.S. Publication 2017/0226117A1, each of which are incorporated by reference in their entirety.

Still further examples of PDE1 inhibitors suitable for use in the methods and treatments discussed herein can be found in International Publication WO2018007249A1; U.S. Publication 2018/0000786; International Publication WO2015118097A1; U.S. Pat. No. 9,718,832; International Publication WO2015091805A1; U.S. Pat. No. 9,701,665; U.S. Publication 2015/0175584A1; U.S. Publication 2017/0267664A1; International Publication WO2016055618A1; U.S. Publication 2017/0298072A1; International Publication WO2016170064A1; U.S. Publication 2016/0311831A1; International Publication WO2015150254A1; U.S. Publication 2017/0022186A1; International Publication WO2016174188A1; U.S. Publication 2016/0318939A1; U.S. Publication 2017/0291903A1; International Publication WO2018073251A1; International Publication WO2017178350A1; U.S. Publication 2017/0291901A1; International Publication WO2018/115067; U.S. Publication 2018/0179200A; U.S. Publication US20160318910A1; U.S. Pat. No. 9,868,741; International Publication WO2017/139186A1; International Application WO2016/040083; U.S. Publication 2017/0240532; International Publication WO 2016033776A1; U.S. Publication 2017/0233373; International Publication WO2015130568; International Publication WO2014159012; U.S. Pat. Nos. 9,034,864; 9,266,859; International Publication WO2009085917; U.S. Pat. No. 8,084,261; International Publication WO2018039052; U.S. Publication US20180062729; and International Publication WO2019027783 each of which are incorporated by reference in their entirety. In any situation in which the statements of any documents incorporated by reference contradict or are incompatible with any statements made in the present disclosure, the statements of the present disclosure shall be understood as controlling.

Still further examples of PDE1 inhibitors and suitable methods of use are disclosed in International Application PCT/US2019/033941 and U.S. Provisional Application 62/789,499, both of which are incorporated by reference herein.

If not otherwise specified or clear from context, the following terms herein have the following meanings:
(a) “Selective PDE1 inhibitor” as used herein refers to a PDE1 inhibitor with at least 100-fold selectivity for PDE1 inhibition over inhibition of any other PDE isoform.
(b) “Alkyl” as used herein is a saturated or unsaturated hydrocarbon moiety, preferably saturated, preferably having one to six carbon atoms, which may be linear or branched, and may be optionally mono-, di- or tri-substituted, e.g., with halogen (e.g., chloro or fluoro), hydroxy, or carboxy.
(c) “Cycloalkyl” as used herein is a saturated or unsaturated nonaromatic hydrocarbon moiety, preferably saturated, preferably comprising three to nine carbon atoms, at least some of which form a nonaromatic mono- or bicyclic, or bridged cyclic structure, and which may be optionally substituted, e.g., with halogen (e.g., chloro or fluoro), hydroxy, or carboxy. Wherein the cycloalkyl optionally contains one or more atoms selected from N and O and/or S, said cycloalkyl may also be a heterocycloalkyl.
(d) “Heterocycloalkyl” is, unless otherwise indicated, saturated or unsaturated nonaromatic hydrocarbon moiety, preferably saturated, preferably comprising three to nine carbon atoms, at least some of which form a nonaromatic mono- or bicyclic, or bridged cyclic structure, wherein at least one carbon atom is replaced with N, O or S, which heterocycloalkyl may be optionally substituted, e.g., with halogen (e.g., chloro or fluoro), hydroxy, or carboxy.
(e) “Aryl” as used herein is a mono or bicyclic aromatic hydrocarbon, preferably phenyl, optionally substituted, e.g., with alkyl (e.g., methyl), halogen (e.g., chloro or fluoro), haloalkyl (e.g., trifluoromethyl), hydroxy, carboxy, or an additional aryl or heteroaryl (e.g., biphenyl or pyridylphenyl).
(f) “Heteroaryl” as used herein is an aromatic moiety wherein one or more of the atoms making up the aromatic ring is sulfur or nitrogen rather than carbon, e.g., pyridyl or thiadiazolyl, which may be optionally substituted, e.g., with alkyl, halogen, haloalkyl, hydroxy or carboxy.

Compounds of the Disclosure, e.g., PDE1 inhibitors as described herein, may exist in free or salt form, e.g., as acid addition salts. In this specification unless otherwise indicated, language such as “Compounds of the Disclosure” is to be understood as embracing the compounds in any form, for example free or acid addition salt form, or where the compounds contain acidic substituents, in base addition salt form. The Compounds of the Disclosure are intended for use as pharmaceuticals, therefore pharmaceutically acceptable salts are preferred. Salts which are unsuitable for pharmaceutical uses may be useful, for example, for the isolation or purification of free Compounds of the Disclosure or their pharmaceutically acceptable salts, are therefore also included.

Compounds of the Disclosure may in some cases also exist in prodrug form. A prodrug form is compound which converts in the body to a Compound of the Disclosure. For example, when the Compounds of the Disclosure contain hydroxy or carboxy substituents, these substituents may form physiologically hydrolysable and acceptable esters. As used herein, “physiologically hydrolysable and acceptable ester” means esters of Compounds of the Disclosure which are hydrolysable under physiological conditions to yield acids (in the case of Compounds of the Disclosure which have hydroxy substituents) or alcohols (in the case of Compounds of the Disclosure which have carboxy substituents) which are themselves physiologically tolerable at doses to be administered. Therefore, wherein the Compound of the Disclosure contains a hydroxy group, for example, Compound-OH, the acyl ester prodrug of such compound, i.e., Compound-O—C(O)—C₁₋₄alkyl, can hydrolyze in the body to form physiologically hydrolysable alcohol (Compound-OH) on the one hand and acid on the other (e.g., HOC(O)—C₁₋₄alkyl). Alternatively, wherein the Compound of the Disclosure contains a carboxylic acid, for example, Compound-C(O)OH, the acid ester prodrug of such compound, Compound-C(O)O—C₁₋₄alkyl can hydrolyze to form Compound-C(O)OH and HO—C₁₋₄alkyl. As will be appreciated the term thus embraces conventional pharmaceutical prodrug forms.

In another embodiment, the disclosure further provides a pharmaceutical composition comprising a PDE1 inhibitor in combination with an agent that increases circulating glucagon (e.g., glucagon and/or sodium glucose cotransporter 2 (SGLT2) inhibitor), each in free or pharmaceutically acceptable salt form, in admixture with a pharmaceutically acceptable carrier. The term “combination,” as used herein, embraces simultaneous, sequential, or contemporaneous administration of the PDE1 inhibitor and the agent that increases circulating glucagon. In another embodiment, the disclosure provides a pharmaceutical composition containing such a compound. In some embodiments, the combination of the PDE1 inhibitor and the agent that increases circulating glucagon allows the agent to be administered in a dosage lower than would be effective if administered as sole monotherapy.

In another embodiment, the disclosure further provides a pharmaceutical composition comprising a Compound of the Disclosure, in free or pharmaceutically acceptable salt form, in admixture with a pharmaceutically acceptable carrier.

In another embodiment, the disclosure further provides a pharmaceutical composition comprising a Compound of the Disclosure, in free, pharmaceutically acceptable salt or prodrug form, in admixture with a pharmaceutically acceptable carrier.

In some embodiments, the Compounds of the Disclosure may be modified to affect their rate of metabolism, e.g., to increase half-life in vivo. In some embodiments, the compounds may be deuterated or fluorinated to reduce the rate of metabolism of the compounds disclosed herein.

In still another further embodiment, the compounds disclosed herein may be in the form of a pharmaceutical composition, for example for oral administration, e.g., in the form of tablets or capsules, or for parenteral administration. In some embodiments, the compounds are provided in the form of a long acting depot composition for administration by injection to provide sustained release. In some embodiments, the solid drug for oral administration or as a depot may be in a suitable polymer matrix to provide delayed release of the active compound.

The Compounds of the Disclosure and their pharmaceutically acceptable salts may be made using the methods as described and exemplified herein and by methods similar thereto and by methods known in the chemical art. If not commercially available, starting materials for these processes may be made by procedures, which are selected from the chemical art using techniques which are similar or analogous to the synthesis of known compounds. Starting materials and methods of making Compounds of the Disclosure are described in the patent applications cited and incorporated by reference above.

The Compounds of the Disclosure include their enantiomers, diastereoisomers and racemates, as well as their polymorphs, hydrates, solvates and complexes. Some individual compounds within the scope of this disclosure may contain double bonds. Representations of double bonds in this disclosure are meant to include both the E and the Z isomer of the double bond. In addition, some compounds within the scope of this disclosure may contain one or more asymmetric centers. This disclosure includes the use of any of the optically pure stereoisomers as well as any combination of stereoisomers.

It is also intended that the Compounds of the Disclosure encompass their stable and unstable isotopes. Stable isotopes are nonradioactive isotopes which contain one additional neutron compared to the abundant nuclides of the same species (i.e., element). It is expected that the activity of compounds comprising such isotopes would be retained, and such compound would also have utility for measuring pharmacokinetics of the non-isotopic analogs. For example, the hydrogen atom at a certain position on the Compounds of the Disclosure may be replaced with deuterium (a stable isotope which is non-radioactive). Examples of known stable isotopes include, but not limited to, deuterium, ¹³C, ¹⁵N, ¹⁸O. Alternatively, unstable isotopes, which are radioactive isotopes which contain additional neutrons compared to the abundant nuclides of the same species (i.e., element), e.g., ¹²³I, ¹³¹I, ¹²⁵I, ¹¹C, ¹⁸F, may replace the corresponding abundant species of I, C and F. Another example of useful isotope of the compound of the disclosure is the ¹¹C isotope. These radio isotopes are useful for radio-imaging and/or pharmacokinetic studies of the compounds of the disclosure.

The present disclosure further provides for inhibitors of sodium glucose cotransporter 2 (SGLT2). Many SGLT2 inhibitors are known, of a variety of chemical structures. For example, the SGLT2 inhibitors of the present disclosure include atigliflozin, canagliflozin, dapagliflozin, empagliflozin, ertugliflozin, ipragliflozin, luseogliflozin, remogliflozin (e.g., remogliflozin etabonate), sergliflozin (e.g., sergliflozin etabonate), sotagliflozin, tofogliflozin, and phlorizin.

In some embodiments, the SGLT2 inhibitors employed in the present disclosure are selective for SGLT2 relative to SGLT1, e.g., dapagliflozin. SGLT2 inhibitors suitable for use in accordance with the present disclosure comprise C-arylglucosides or O-arylglucosides. C-arylglucosides and O-arylglucosides are effective in treating diabetes. See U.S. Pat. No. 6,774,112, which is incorporated herein by reference in its entirety. Examples of C-arylglucoside (also referred to as C-glucosides) SGLT2 inhibitors which can be employed in the methods of the disclosure, include those disclosed in U.S. Pat. Nos. 6,515,117, 6,414,126, and 6,774,112, as well as U.S. Publications US2006/0063722, US2005/0209166, and US2006/0074031, the disclosures of each of which are incorporated herein by reference in their entireties. Examples of O-glucoside SGLT2 inhibitors which can be employed in the methods of the disclosure include those disclosed in U.S. Pat. Nos. 6,908,905 and 6,815,428, U.S. Publication US2006/0194809, as well as International Publication WO 03/01180, the disclosures of each of which are incorporated herein by reference in their entireties. Further examples of SGLT2 inhibitors are disclosed in WO2001/027128, WO2003/099836, WO2008/002824, WO2006/034489, US2005/0209166, US2007/0054867, US2005/0209166, WO2006/117360, US2009/0118201, US2008/0113922, US2009/0030198, WO2005/012326, WO2004013118, WO2005/012326, WO2008/069327, WO2009/035969, US2008/0132563, WO2005/092877, WO2006/064033, WO2006/117359, WO2007/025943, WO2007/028814, WO2007/031548, WO2007/093610, WO2007/128749, WO2008/049923, WO2008/055870, WO2008/055940, WO2009/022020, WO2009/022008, WO2014/016381, WO2006/120208, WO2011/039108, WO2004/007517, WO2004/080990, WO2007/114475, WO2010/023594, WO2007/140191, WO2008/013280, the disclosures of each of which are incorporated herein by reference in their entireties.

Other disclosures and publications disclosing SGLT2 inhibitors that can be employed in the methods of the disclosure are as follows: K. Tsujihara et al., Chem. Pharm. Bull., 44:1174-1180 (1996); M. Hongu et al., Chem. Pharm. Bull., 46:22-33 (1998); M. Hongu et al., Chem. Pharm. Bull., 46:1545-1555 (1998); and A. Oku et al., Diabetes, 48:1794-1800 (1999) and JP 10245391 (Dainippon), the disclosures of each of which are incorporated herein by reference in their entireties.

In some embodiments, the present disclosure provides SGLT2 inhibitors for use in the methods of the disclosure that are disclosed in U.S. Pat. Nos. 6,414,126 and 6,515,117, e.g., the SGLT2 inhibitor is dapagliflozin

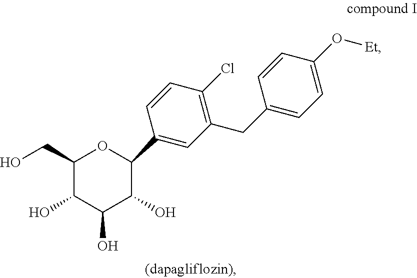 [SMILES: CCOc1ccc(Cc2cc([C@@H]3O[C@H](CO)[C@@H](O)[C@H](O)[C@H]3O)ccc2Cl)cc1]

or a pharmaceutically acceptable salt thereof, all stereoisomers thereof, or a prodrug ester thereof.

In other embodiments, the disclosure provides crystalline forms of compound I including the crystalline forms disclosed in U.S. Patent Application Publication No. 2008/0004336, the disclosure of which is incorporated herein by reference in its entirety.

Additional SGLT2 inhibitors that may be employed in the present disclosure include canagliflozin (Johnson & Johnson/Mitsubishi Tanabe Pharma); remogliflozin etabonate (Islet Sciences, Kissei Pharmaceuticals Co.); ipragliflozin (Astellas/Kotobuki); empagliflozin (Boehringer Ingelheim); BI-44847 (Boehringer Ingelheim); TS-071 (Taisho Pharmaceutical); tofogliflozin (Roche/Chugai Pharmaceutical); LX-4211 (Lexicon Pharmaceuticals); DSP-3235 (GlaxoSmithKline/Dainippon Sumitomo); ISIS-SGLT2Rx (Isis Pharmaceuticals); and YM543 (Astellas Pharma Inc). A further SGLT-2 inhibitor is ertugliflozin (Pfizer and Merck).

Various forms of prodrugs are known in the art. Examples of such prodrugs are disclosed in, for example, Design of Prodrugs, edited by H. Bundgaard, (Elsevier, 1985) and Methods in Enzymology, Vol. 42, p. 309-396, edited by K. Widder, et al. (Academic Press, 1985); A Textbook of Drug Design and Development, edited by Krogsgaard-Larsen and H. Bundgaard, Chapter 5 “Design and Application of Prodrugs”, by H. Bundgaard p. 113-191 (1991); and H. Bundgaard, Advanced Drug Delivery Reviews, 8, 1-38 (1992); d) H. Bundgaard, et al., Journal of Pharmaceutical Sciences, 77, 285 (1988); and e) N. Kakeya, et al., Chem Pharm Bull, 32, 692 (1984).

Examples of such prodrugs are in vivo cleavable esters of a compound of the disclosure. An in vivo cleavable ester of a compound of the disclosure containing a carboxy group is, for example, a pharmaceutically-acceptable ester which is cleaved in the human or animal body to produce the parent acid. Suitable pharmaceutically-acceptable esters for carboxy include (1-6C)alkyl esters, for example methyl or ethyl; (1-6C)alkoxymethyl esters, for example methoxymethyl; (1-6C)alkanoyloxymethyl esters, for example pivaloyloxymethyl; phthalidyl esters; (3-8C)cycloalkoxycarbonyloxy(1-6C)alkyl esters, for example 1-cyclohexylcarbonyloxyethyl; 1,3-dioxolan-2-ylmethyl esters, for example 5-methyl-1,3-dioxolan-2-ylmethyl; (1-6C)alkoxycarbonyloxyethyl esters, for example 1-methoxycarbonyloxyethyl; aminocarbonylmethyl esters and mono- or di-N-((1-6C)alkyl) versions thereof, for example N,N-dimethylaminocarbonylmethyl esters and N-ethylaminocarbonylmethyl esters; and may be formed at any carboxy group in the compounds of this disclosure. An in vivo cleavable ester of a compound of the disclosure containing a hydroxy group is, for example, a pharmaceutically-acceptable ester which is cleaved in the human or animal body to produce the parent hydroxy group. Suitable pharmaceutically acceptable esters for hydroxy include (1-6C)alkanoyl esters, for example acetyl esters; and benzoyl esters wherein the phenyl group may be substituted with aminomethyl or N-substituted mono- or di-(1-6C)alkyl aminomethyl, for example 4-aminomethylbenzoyl esters and 4-N,N-dimethylaminomethylbenzoyl esters.

### Methods of Using Compounds of the Disclosure

In various embodiments, the present disclosure provides a method [Method 1] for the treatment or prophylaxis of a disease, disorder or condition associated with altered glucagon function and/or altered cyclic nucleotides (e.g., cAMP and/or cGMP) signaling, comprising administration of a pharmaceutically effective amount of a PDE1 inhibitor (e.g., a PDE1 inhibitor of Formula I, Ia, II, III, IV, V, VI and/or VII as herein described) and a pharmaceutically effective amount of an agent that increases circulating glucagon levels or activates glucagon receptors (e.g., glucagon, SGLT2 inhibitor or other agents that may directly or indirectly increase plasma glucagon) to a patient in need thereof. For example, the present disclosure provides for the following embodiments of Method 1:
1.1 Method 1, wherein the disease, disorder or condition is associated with altered cyclic nucleotides (e.g., cAMP or cGMP) signaling.
1.2 Any of the preceding methods, wherein the disease, disorder or condition is associated with altered glucagon function.
1.3 Any of the preceding methods, wherein the disease, disorder or condition is associated with altered cyclic nucleotides (e.g., cAMP and/or cGMP) signaling and altered glucagon function.
1.4 Any of the preceding methods, wherein the condition, disease or disorder is a cardiovascular condition, disease or disorder, a renal condition, disease or disorder, or a metabolic condition, disease or disorder.
1.5 Any of the preceding methods, wherein the condition, disease or disorder is a cardiovascular condition, disease or disorder selected from angina, stroke, renal failure, essential hypertension, pulmonary hypertension, secondary hypertension, isolated systolic hypertension, hypertension associated with diabetes, hypertension associated with atherosclerosis, renovascular hypertension, congestive heart failure, an inflammatory disease or disorder, fibrosis (e.g., cardiac fibrosis associated with or induced by ventricular or supraventricular fibrillation), cardiac remodeling during gestation (i.e., peripartum cardiomyopathy), cardiac hypertrophy, vascular remodeling, a connective tissue disease or disorder (e.g., Marfan Syndrome), chronic heart failure, acute heart failure, heart failure consequent to myocardial infarction, myocardial ischemia, myocardial hypoxia, reperfusion injury, left ventricular dysfunctions (e.g., myocardial infarction, ventricular expansion), vascular leakage (i.e., consequent to hypoxia), muscular dystrophy (e.g., Duchenne muscular dystrophy), and/or amyotrophic lateral sclerosis.
1.6 Any of the preceding methods, wherein the condition, disease or disorder is heart failure (e.g., chronic heart failure, acute heart failure, heart failure consequent to myocardial infarction, congestive heart failure).
1.7 Any of the preceding methods, wherein the condition, disease or disorder is chronic heart failure.
1.8 Any of the preceding methods, wherein the condition, disease or disorder is acute heart failure.
1.9 Any of the preceding methods, wherein the condition, disease or disorder is heart failure consequent to myocardial infarction.
1.10 Any of the preceding methods, wherein the condition, disease or disorder is congestive heart failure.
1.11 Any of the preceding methods, wherein the condition, disease or disorder is hypertension (e.g., essential hypertension, pulmonary hypertension, secondary hypertension, isolated systolic hypertension, hypertension associated with diabetes, hypertension associated with atherosclerosis, renovascular hypertension).
1.12 Any of the preceding methods, wherein the condition, disease or disorder is essential hypertension.
1.13 Any of the preceding methods, wherein the condition, disease or disorder is pulmonary hypertension.
1.14 Any of the preceding methods, wherein the condition, disease or disorder is secondary hypertension.
1.15 Any of the preceding methods, wherein the condition, disease or disorder is isolated systolic hypertension.
1.16 Any of the preceding methods, wherein the condition, disease or disorder is hypertension associated with diabetes.
1.17 Any of the preceding methods, wherein the condition, disease or disorder is hypertension associated with atherosclerosis.
1.18 Any of the preceding methods, wherein the condition, disease or disorder is renovascular hypertension.
1.19 Any of the preceding methods, wherein the condition, disease or disorder is a renal condition, disease or disorder (e.g., kidney fibrosis, chronic kidney disease, renal failure, glomerulosclerosis and nephritis, a renal disorder consequent to diabetes, an injury to a kidney, high blood pressure, a cancerous growth (e.g., polycystic kidney disease)).
1.20 Any of the preceding methods, wherein the condition, disease or disorder is kidney fibrosis.
1.21 Any of the preceding methods, wherein the condition, disease or disorder is chronic kidney disease.
1.22 Any of the preceding methods, wherein the condition, disease or disorder is renal failure.
1.23 Any of the preceding methods, wherein the condition, disease or disorder is a renal condition, disease or disorder consequent to diabetes mellitus.
1.24 Any of the preceding methods, wherein the condition, disease or disorder is a metabolic disease, disorder or condition (e.g., type 1 diabetes mellitus, type 2 diabetes mellitus, high cholesterol, impaired glucose tolerance (IGT), impaired fasting blood glucose (IFG), hyperglycemia, postprandial hyperglycemia, overweight, obesity, metabolic syndrome, gestational diabetes, insulin resistance, conditions consequent to diabetes mellitus, e.g., cataracts and micro- and macrovascular diseases, such as nephropathy, retinopathy, neuropathy, tissue ischemia, arteriosclerosis, myocardial infarction, stroke and peripheral arterial occlusive disease, degeneration or decline in the functionality of pancreatic beta cells, conditions consequent to an abnormal accumulation of ectopic fat, hyperinsulemia, new onset diabetes after transplantation (NODAT), post-transplant metabolic syndrome (PTMS), focal segmented glomerulosclerosis, conditions consequent to NODAT and/or PTMS (e.g., micro- and macrovascular diseases and events, graft rejection, infection, and death), hyperuricemia, kidney stones, hyponatremia, peripheral fluid retention and peripheral edema).
1.25 Any of the preceding methods, wherein the condition, disease or disorder is type 1 diabetes mellitus.
1.26 Any of the preceding methods, wherein the condition, disease or disorder is type 2 diabetes mellitus.
1.27 Any of the preceding methods, wherein the condition, disease or disorder is gestational diabetes.
1.28 Any of the preceding methods, wherein the condition, disease or disorder is impaired glucose tolerance.
1.29 Any of the preceding methods, wherein the condition, disease or disorder is hyperglycemia.
1.30 Any of the preceding methods, wherein the condition, disease or disorder is a condition consequent to diabetes mellitus (e.g., cataracts and micro- and macrovascular diseases, such as nephropathy, retinopathy, neuropathy, tissue ischemia, arteriosclerosis, myocardial infarction, stroke and peripheral arterial occlusive disease).
1.31 Any of the preceding methods, wherein the PDE1 inhibitor is administered at a concentration of 0.01 mg/kg to 100 mg/kg.
1.32 Any of the preceding methods wherein the patient is a human and the PDE1 inhibitor is administered at an oral daily dosage of 1-300 mg.
1.33 Any of the preceding methods, wherein the PDE1 inhibitor is administered at a dosage of 1 mg, 3, mg, 10 mg, 30 mg, or 90 mg.
1.34 Any of the preceding methods, wherein the agent that increases circulating glucagon levels or activates glucagon receptors (e.g., glucagon, SGLT2 inhibitor or other agents that may directly or indirectly increase plasma glucagon) is administered at a dosage of 0.001 mg/kg to about 100 mg/kg body weight of the subject.
1.35 Any of the preceding methods, wherein the PDE1 inhibitor is administered orally.
1.36 Any of the preceding methods, wherein the PDE1 inhibitor is administered as a tablet or capsule.
1.37 Any of the preceding methods, wherein the PDE1 inhibitor is a compound according to any of Formulas I, Ia, II, III, IV, V, VI and/or VII.
1.38 Any of the preceding methods, wherein the PDE1 inhibitor is a compound according to Formula Ia.
1.39 Any of the preceding methods, wherein the PDE1 inhibitor is a compound according to:

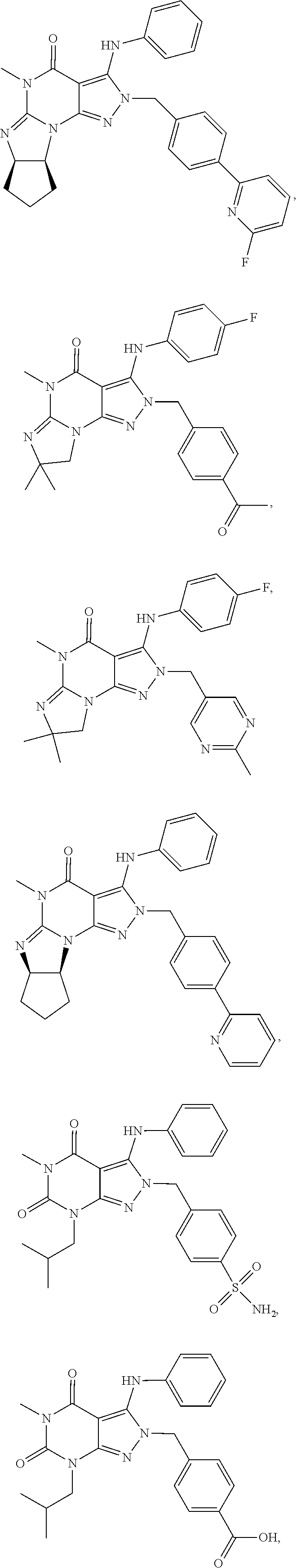

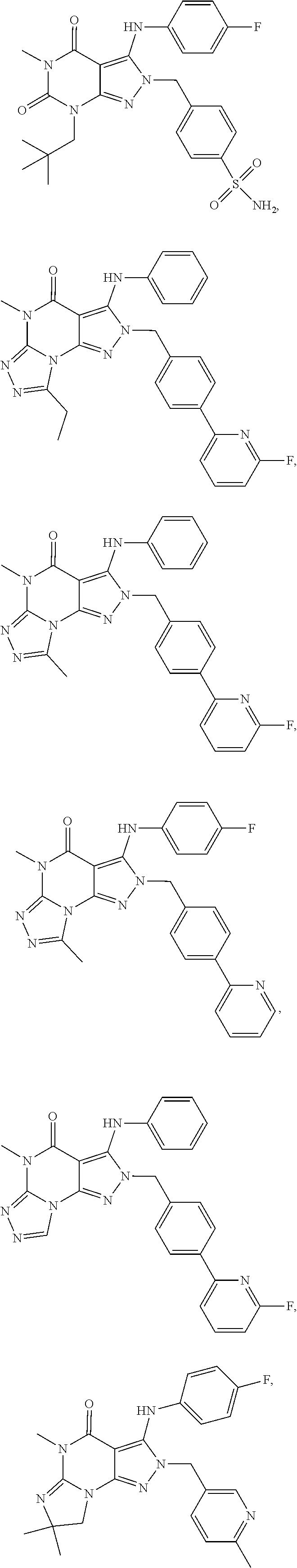

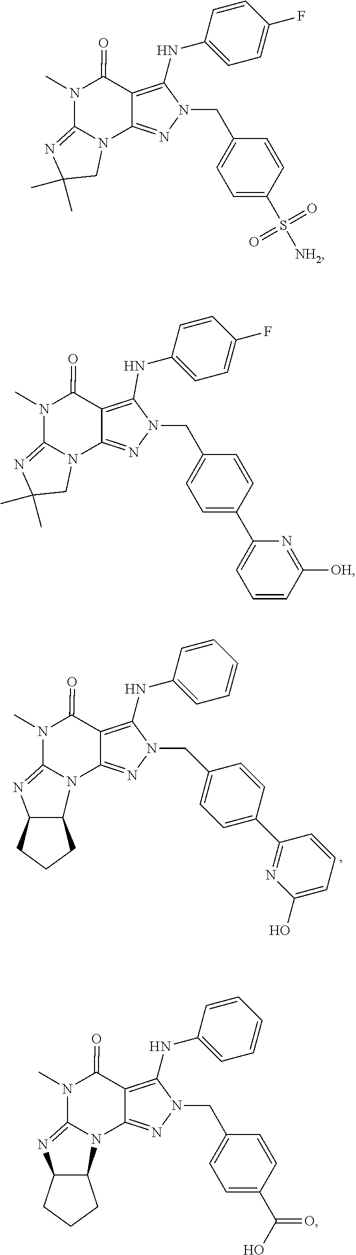 [SMILES: CN1C(=O)c2c(nn(Cc3ccc(S(N)(=O)=O)cc3)c2Nc2ccc(F)cc2)N2CC(C)(C)N=C12 ; CN1C(=O)c2c(nn(Cc3ccc(-c4cccc(O)n4)cc3)c2Nc2ccc(F)cc2)N2CC(C)(C)N=C12 ; CN1C(=O)c2c(nn(Cc3ccc(-c4cccc(O)n4)cc3)c2Nc2ccccc2)N2C1=N[C@@H]1CCC[C@@H]12 ; CN1C(=O)c2c(nn(Cc3ccc(C(=O)O)cc3)c2Nc2ccccc2)N2C1=N[C@@H]1CCC[C@@H]12]

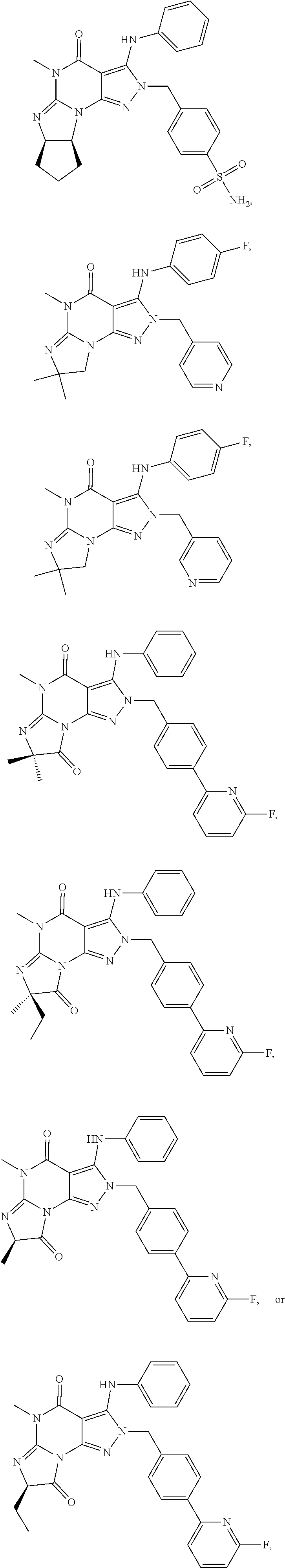

in free or pharmaceutically acceptable salt form.
1.40 Any of the preceding methods, wherein the PDE1 inhibitor is a compound according to:

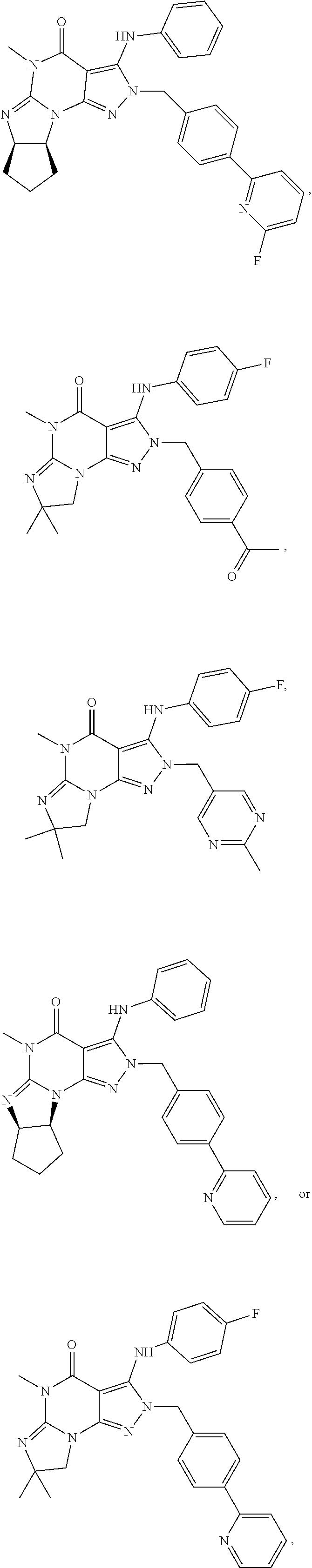

in free or pharmaceutically acceptable salt form.
1.41 Any of the preceding methods, wherein the PDE1 inhibitor is a compound according to:

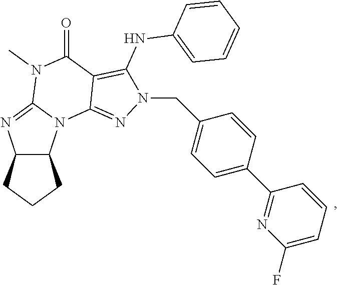 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.
1.42 Any of the preceding methods, wherein the PDE1 inhibitor is a compound according to:

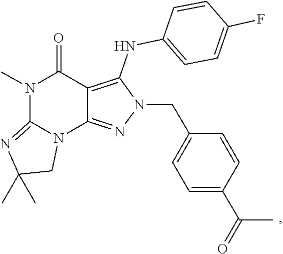 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.
1.43 Any of the preceding methods, wherein the PDE1 inhibitor is a compound according to:

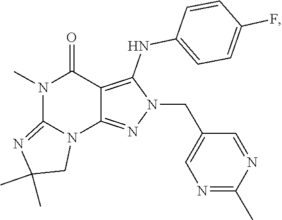 [SMILES: Cc1ncc(Cn2nc3c(c2Nc2ccc(F)cc2)C(=O)N(C)C2=NC(C)(C)CN23)cn1]

in free or pharmaceutically acceptable salt form.
1.44 Any of the preceding methods, wherein the PDE1 inhibitor is a compound according to:

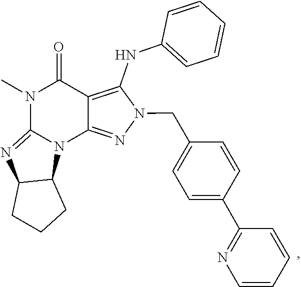 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.
1.45 Any of the preceding methods, wherein the PDE1 inhibitor is a compound according to:

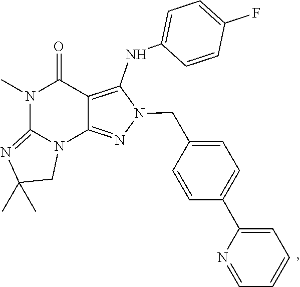 [SMILES: CN1C(=O)c2c(nn(Cc3ccc(-c4ccccn4)cc3)c2Nc2ccc(F)cc2)N2CC(C)(C)N=C12]

in free or pharmaceutically acceptable salt form.
1.46 Any of the preceding methods wherein the patient is a human, the PDE1 inhibitor is administered at an oral daily dosage of 0.01-300 mg, e.g., 1-90 mg, e.g., 1 mg, 3, mg, 10 mg, 30 mg, or 90 mg, and the PDE1 inhibitor is selected from
   a. (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one, in free or pharmaceutically acceptable salt form, e.g., monophosphate salt form;
   b. 7,8-dihydro-2-(4-acetylbenzyl)-3-(4-fluorophenylamino)-5,7,7-trimethyl-[2H]-imidazo-[1,2-a]pyrazolo[4,3-e]pyrimidin-4(5H)-one, in free or pharmaceutically acceptable salt form; and
   c. 3-((4-fluorophenyl)amino)-5,7,7-trimethyl-2-((2-methylpyrimidin-5-yl)methyl)-7,8-dihydro-2H-imidazo[1,2-a]pyrazolo[4,3-e]pyrimidin-4(5H)-one, in free or pharmaceutically acceptable salt form.
1.47 Any of the preceding methods, wherein the patient is a human.
1.48 Any of the preceding methods, wherein the agent that modulates circulating glucagon levels or activates glucagon receptors is selected from glucagon, atigliflozin, canagliflozin, dapagliflozin, empagliflozin, ertugliflozin, ipragliflozin, luseogliflozin, remogliflozin (e.g., remogliflozin etabonate), sergliflozin (e.g., sergliflozin etabonate), sotagliflozin, tofogliflozin, and phlorizin, in free or pharmaceutically acceptable salt form.
1.49 Any of the preceding methods, wherein the agent that modulates circulating glucagon levels or activates glucagon receptors is selected from glucagon, canagliflozin, dapagliflozin, empagliflozin, ertugliflozin, in free or pharmaceutically acceptable salt form.
1.50 Any of the preceding methods, wherein the agent that increases circulating glucagon levels or activates glucagon receptors is dapagliflozin, in free or pharmaceutically acceptable salt form.
1.51 Any of the preceding methods, wherein the agent that increases circulating glucagon levels or activates glucagon receptors is dapagliflozin in free form.
1.52 Any of the preceding methods, wherein the agent that increases circulating glucagon levels or activates glucagon receptors is glucagon.
1.53 Any of the preceding methods, further comprising administering a pharmaceutically effective amount of a dipeptidyl peptidase-4 (DPP-4) inhibitor.
1.54 The preceding method, wherein the DPP-4 inhibitor comprises sitagliptin, vildagliptin, saxagliptin, linagliptin, gemigliptin, anagliptin, teneligliptin, alogliptin, trelagliptin, omarigliptin, evogliptin, gosogliptin, and/or dutogliptin, in free or pharmaceutically acceptable salt form.
1.55 Any of the preceding methods, wherein the patient is already being administered a DPP-4 inhibitor.

The disclosure further provides a PDE1 inhibitor and an agent that increases modulates glucagon levels or activates glucagon receptors (e.g., glucagon, SGLT2 inhibitor or other agents that may directly or indirectly increase plasma glucagon) for use in a method of treating a disease, disorder or condition associated with altered glucagon function and/or a disease, disorder or condition mediated by altered cyclic nucleotides (e.g., cAMP and/or cGMP) signaling, e.g., for use in any of Methods 1, et seq.

The disclosure further provides the use of a combination therapy comprising or consisting of a PDE1 inhibitor and an agent that increases circulating glucagon levels or activates glucagon receptors (e.g., glucagon, SGLT2 inhibitor or other agents that may directly or indirectly increase plasma glucagon) in the manufacture of a medicament for use in a method of treating a disease, disorder or condition associated with altered glucagon function and/or a disease, disorder or condition mediated by altered cyclic nucleotides (e.g., cAMP and/or cGMP) signaling, e.g., a medicament for use in any of Methods 1, et seq.

The disclosure further provides a pharmaceutical composition comprising a PDE1 inhibitor, e.g., any of a Compound of Formulas I, Ia, II, III, IV, V, VI and/or VII, and a pharmaceutically effective amount of an agent that increases circulating glucagon levels or activates glucagon receptors (e.g., glucagon, SGLT2 inhibitor or other agents that may directly or indirectly increase plasma glucagon) for use in any of Methods 1, et seq.

Combination Therapies with PDEI Inhibitors

In some embodiments, the PDE1 inhibitor is administered in combination with other therapeutic modalities. For example, a patient may be administered with agent that increases circulating glucagon levels or activates glucagon receptors (e.g., glucagon, SGLT2 inhibitor or other agents that may directly or indirectly increase plasma glucagon) in combination with any of the disclosed PDE1 inhibitors.

Combinations may be achieved by administering a single composition or pharmacological formulation that includes the PDE1 inhibitor and one or more additional therapeutic agents, or by administration of two distinct compositions or formulations, separately, simultaneously or sequentially, wherein one composition includes the PDE1 inhibitor and the other includes the additional therapeutic agent or agents. The therapy using a PDE1 inhibitor may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In some embodiments, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a PDE1 inhibitor, or an additional therapeutic agent will be desired. In this regard, various combinations may be employed. By way of illustration, where the PDE1 inhibitor is “A” and the additional therapeutic agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Accordingly, in various embodiments, the present disclosure also provides for a pharmaceutical combination [Combination 1] therapy comprising a pharmaceutically effective amount of a PDE1 inhibitor (e.g., a compound according to any of Formula I, II, III, IV, V, VI and/or VII) and a pharmaceutically effective amount of an agent that increases circulating glucagon levels or activates glucagon receptors (e.g., glucagon, SGLT2 inhibitor or other agents that may directly or indirectly increase plasma glucagon), for administration in a method of treating a disease, disorder or condition associated with altered glucagon function and/or a disease, disorder or condition mediated by altered cyclic nucleotides (e.g., cAMP and/or cGMP) signaling, e.g., in accordance with any of Method 1, et seq. For example, the present disclosure provides for the following Combinations:
1.1 Combination 1 wherein the PDE1 inhibitor and the agent that increases circulating glucagon levels or activates glucagon receptors are in a single dosage form, e.g., a tablet or capsule, in combination or association with a pharmaceutically acceptable diluent or carrier.
1.2 Any of the preceding combinations, wherein the PDE1 inhibitor and the agent that increases circulating glucagon levels or activates glucagon receptors are in a single package, e.g., with instructions for administration simultaneously or sequentially.
1.3 Any of the preceding combinations, wherein the PDE1 inhibitor is a compound according to any of Formulas I, Ia, II, III, IV, V, VI and/or VII.
1.4 Any of the preceding combinations, wherein the PDE1 inhibitor is a compound according to Formula Ia.
1.5 Any of the preceding combinations, wherein the PDE1 inhibitor is a compound according to:

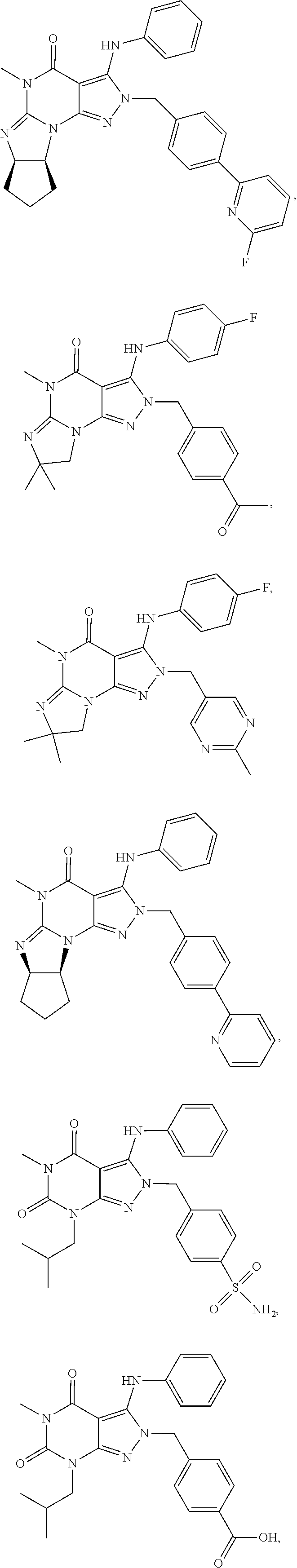

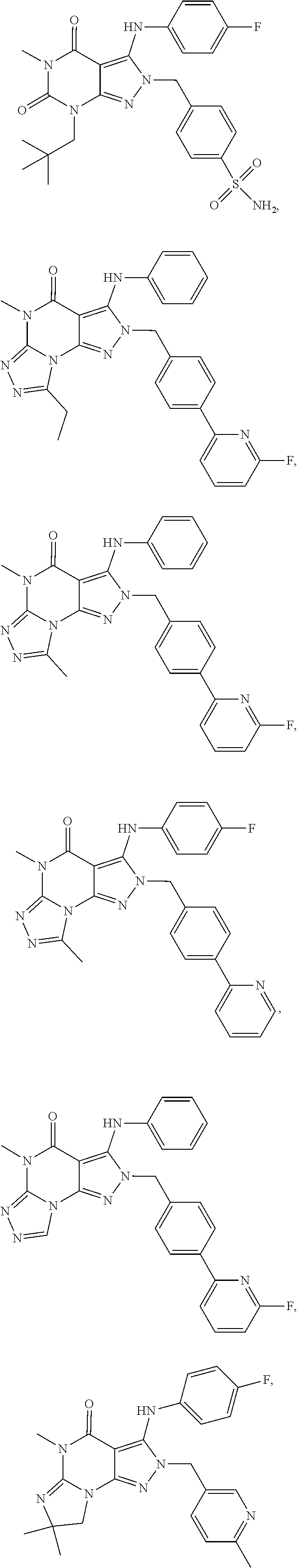

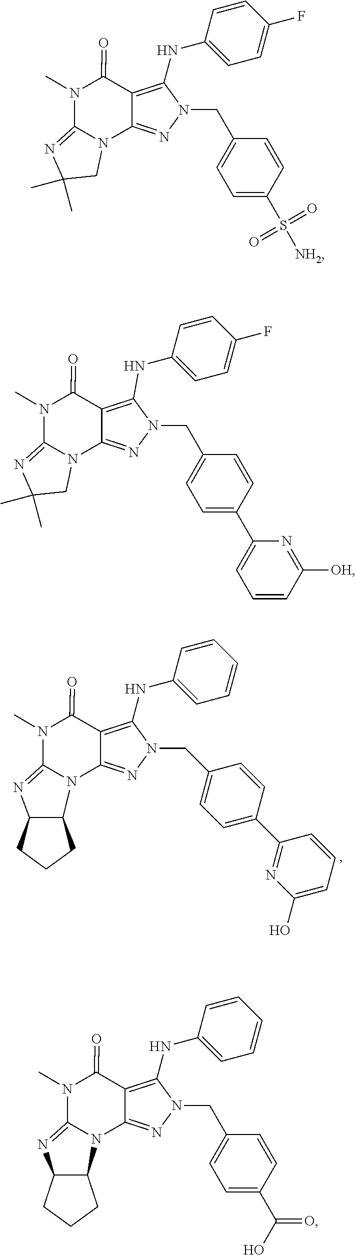 [SMILES: CN1C(=O)c2c(nn(Cc3ccc(S(N)(=O)=O)cc3)c2Nc2ccc(F)cc2)N2CC(C)(C)N=C12 ; CN1C(=O)c2c(nn(Cc3ccc(-c4cccc(O)n4)cc3)c2Nc2ccc(F)cc2)N2CC(C)(C)N=C12 ; CN1C(=O)c2c(nn(Cc3ccc(-c4cccc(O)n4)cc3)c2Nc2ccccc2)N2C1=N[C@@H]1CCC[C@@H]12 ; CN1C(=O)c2c(nn(Cc3ccc(C(=O)O)cc3)c2Nc2ccccc2)N2C1=N[C@@H]1CCC[C@@H]12]

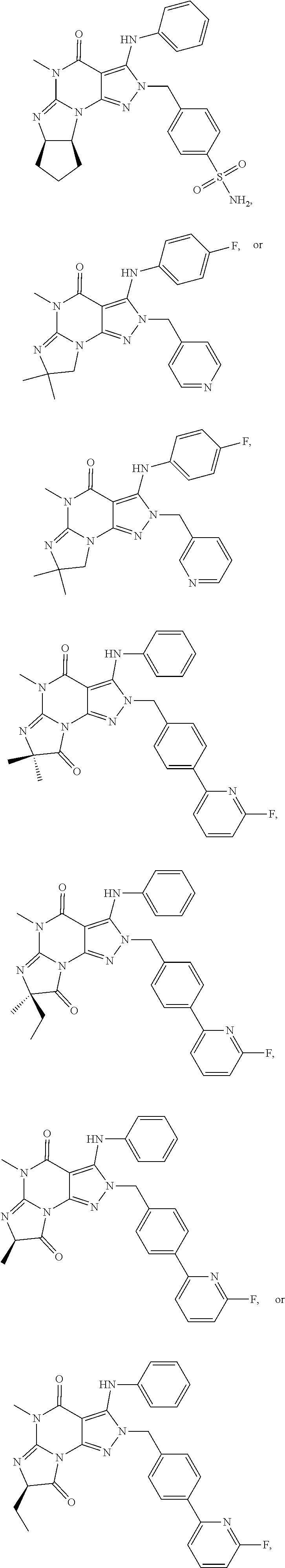

in free or pharmaceutically acceptable salt form.
1.6 Any of the preceding combinations, wherein the PDE1 inhibitor is a compound according to:

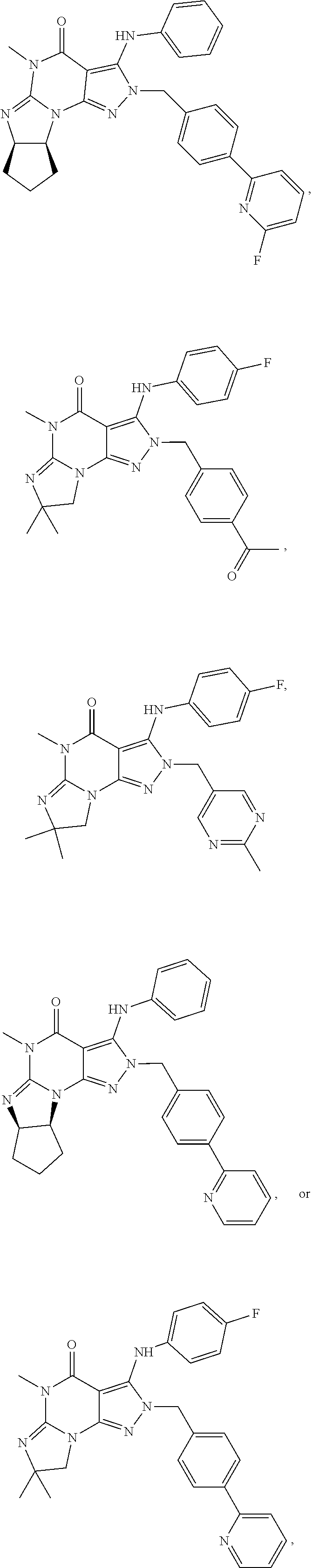

in free or pharmaceutically acceptable salt form.
1.7 Any of the preceding combinations, wherein the PDE1 inhibitor is a compound according to:

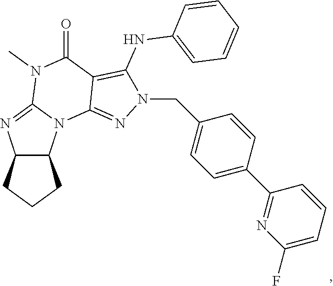 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.
1.8 Any of the preceding combinations, wherein the PDE1 inhibitor is a compound according to:

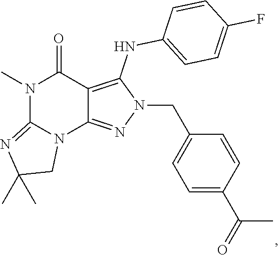 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.
1.9 Any of the preceding combinations, wherein the PDE1 inhibitor is a compound according to:

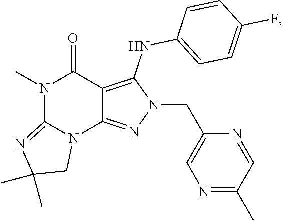 [SMILES: Cc1cnc(Cn2nc3c(c2Nc2ccc(F)cc2)C(=O)N(C)C2=NC(C)(C)CN23)cn1]

in free or pharmaceutically acceptable salt form.
1.10 Any of the preceding combinations, wherein the PDE1 inhibitor is a compound according to:

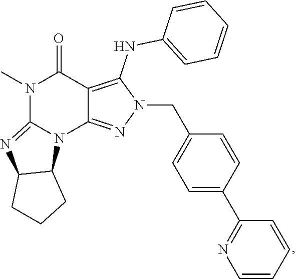 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.
1.11 Any of the preceding combinations, wherein the PDE1 inhibitor is a compound according to:

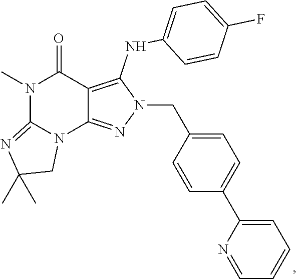 [SMILES: CN1C(=O)c2c(nn(Cc3ccc(-c4ccccn4)cc3)c2Nc2ccc(F)cc2)N2CC(C)(C)N=C12]

in free or pharmaceutically acceptable salt form.
1.12 Any of the preceding combinations wherein the patient is a human, the PDE1 inhibitor is administered at an oral daily dosage of 0.01-300 mg, e.g., 1-90 mg, e.g., 1 mg, 3, mg, 10 mg, 30 mg, or 90 mg, and the PDE1 inhibitor is selected from
   a. (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one, in free or pharmaceutically acceptable salt form, e.g., monophosphate salt form;
   b. 7,8-dihydro-2-(4-acetylbenzyl)-3-(4-fluorophenylamino)-5,7,7-trimethyl-[2H]-imidazo-[1,2-a]pyrazolo[4,3-e]pyrimidin-4(5H)-one, in free or pharmaceutically acceptable salt form; and
   c. 3-((4-fluorophenyl)amino)-5,7,7-trimethyl-2-((2-methylpyrimidin-5-yl)methyl)-7,8-dihydro-2H-imidazo[1,2-a]pyrazolo[4,3-e]pyrimidin-4(5H)-one, in free or pharmaceutically acceptable salt form.
1.13 Any of the preceding combinations, wherein the subject is a human.
1.14 Any of the preceding combinations, wherein the agent that increases circulating glucagon levels or activates glucagon receptors is selected from glucagon, atigliflozin, canagliflozin, dapagliflozin, empagliflozin, ertugliflozin, ipragliflozin, luseogliflozin, remogliflozin (e.g., remogliflozin etabonate), sergliflozin (e.g., sergliflozin etabonate), sotagliflozin, tofogliflozin, and phlorizin, in free or pharmaceutically acceptable salt form.
1.15 Any of the preceding combinations, wherein the agent that increases circulating glucagon levels or activates glucagon receptors is selected from glucagon, canagliflozin, dapagliflozin, empagliflozin, ertugliflozin, in free or pharmaceutically acceptable salt form.
1.16 Any of the preceding combinations, wherein the agent that increases circulating glucagon levels or activates glucagon receptors is dapagliflozin, in free or pharmaceutically acceptable salt form.
1.17 Any of the preceding combinations, wherein the agent that increases circulating glucagon levels or activates glucagon receptors is dapagliflozin in free form.
1.18 Any of the preceding combinations, wherein the agent that increases circulating glucagon levels or activates glucagon receptors is glucagon.
1.19 Any of the preceding combinations, wherein the subject is suffering from a condition, disease or disorder which is mediated by cyclic nucleotides (e.g., cAMP or cGMP).
1.20 Any of the preceding combinations, wherein the subject is suffering from a condition, disease or disorder which is mediated by sodium glucose cotransporter 2 (SGLT2).
1.21 Any of the preceding combinations, wherein the subject is suffering from a condition, disease or disorder which is mediated by cyclic nucleotides (e.g., cAMP or cGMP) and sodium glucose cotransporter 2 (SGLT2).
1.22 Any of the preceding combinations, wherein the subject is suffering from a cardiovascular condition, disease or disorder, a renal condition, disease or disorder, or a metabolic condition, disease or disorder.
1.23 Any of the preceding combinations, further comprising a pharmaceutically effective amount of a dipeptidyl peptidase-4 (DPP-4) inhibitor.
1.24 The preceding combination, wherein the DPP-4 inhibitor comprises sitagliptin, vildagliptin, saxagliptin, linagliptin, gemigliptin, anagliptin, teneligliptin, alogliptin, trelagliptin, omarigliptin, evogliptin, gosogliptin, and/or dutogliptin, in free or pharmaceutically acceptable salt form.

“PDE1 inhibitor” as used herein describes a compound(s) which selectively inhibit phosphodiesterase-mediated (e.g., PDE1-mediated) hydrolysis of cGMP and cAMP, e.g., with an IC₅₀ of less than 1 μM, preferably less than 750 nM, more preferably less than 500 nM, more preferably less than 50 nM in an immobilized-metal affinity particle reagent PDE assay.

The phrase “Compounds of the Disclosure” or “PDE 1 inhibitors of the Disclosure”, or like terms, encompasses any such compounds disclosed herewith, e.g., a Compound of Formula I, Formula II, Formula III, Formula IV, Formula V, Formula VI and/or Formula VII.

The words “treatment” and “treating” are to be understood accordingly as embracing prophylaxis and treatment or amelioration of symptoms of disease as well as treatment of the cause of the disease.

For methods of treatment, the word “effective amount” is intended to encompass a therapeutically effective amount to treat a specific disease or disorder.

The terms “patient” or “subject” includes human or non-human (i.e., animal) patient. In particular embodiment, the disclosure encompasses both human and nonhuman. In another embodiment, the disclosure encompasses nonhuman. In other embodiment, the term encompasses human.

The term “comprising” as used in this disclosure is intended to be open-ended and does not exclude additional, unrecited elements or method steps.

Dosages employed in practicing the present disclosure will of course vary depending, e.g. on the particular disease or condition to be treated, the particular Compound of the Disclosure used, the mode of administration, and the therapy desired. Compounds of the Disclosure may be administered by any suitable route, including orally, parenterally, transdermally, or by inhalation, but are preferably administered orally. In general, satisfactory results, e.g. for the treatment of diseases as hereinbefore set forth are indicated to be obtained on oral administration at dosages of the order from about 0.01 to 2.0 mg/kg. In larger mammals, for example humans, an indicated daily dosage for oral administration will accordingly be in the range of from about 0.75 to 150 mg, conveniently administered once, or in divided doses 2 to 4 times, daily or in sustained release form. Unit dosage forms for oral administration thus for example may comprise from about 0.2 to 75 or 150 mg, e.g. from about 0.2 or 2.0 to 50, 75 or 100 mg of a Compound of the Disclosure, together with a pharmaceutically acceptable diluent or carrier therefor.

Pharmaceutical compositions comprising Compounds of the Disclosure may be prepared using conventional diluents or excipients and techniques known in the galenic art. Thus, oral dosage forms may include tablets, capsules, solutions, suspensions and the like.

### EXAMPLES

### Example 1: Measurement of PDEIB Inhibition In Vitro Using IMAP Phosphodiesterase Assay Kit

Phosphodiesterase I B (PDEIB) is a calcium/calmodulin dependent phosphodiesterase enzyme that converts cyclic guanosine monophosphate (cGMP) to 5′-guanosine monophosphate (5′-GMP). PDEIB can also convert a modified cGMP substrate, such as the fluorescent molecule cGMP-fluorescein, to the corresponding GMP-fluorescein. The generation of GMP-fluorescein from cGMP-fluorescein can be quantitated, using, for example, the IMAP (Molecular Devices, Sunnyvale, CA) immobilized-metal affinity particle reagent.

Briefly, the IMAP reagent binds with high affinity to the free 5′-phosphate that is found in GMP-fluorescein and not in cGMP-fluorescein. The resulting GMP-fluorescein-IMAP complex is large relative to cGMP-fluorescein. Small fluorophores that are bound up in a large, slowly tumbling, complex can be distinguished from unbound fluorophores, because the photons emitted as they fluoresce retain the same polarity as the photons used to excite the fluorescence.

In the phosphodiesterase assay, cGMP-fluorescein, which cannot be bound to IMAP, and therefore retains little fluorescence polarization, is converted to GMP-fluorescein, which, when bound to IMAP, yields a large increase in fluorescence polarization (Amp). Inhibition of phosphodiesterase, therefore, is detected as a decrease in Amp.

### Enzyme Assay

Materials: All chemicals are available from Sigma-Aldrich (St. Louis, MO) except for IMAP reagents (reaction buffer, binding buffer, FL-GMP and IMAP beads), which are available from Molecular Devices (Sunnyvale, CA).

Assay: The following phosphodiesterase enzymes may be used: 3′,5′-cyclic-nucleotide-specific bovine brain phosphodiesterase (Sigma, St. Louis, MO) (predominantly PDEIB) and recombinant full length human PDEl A and PDE1B (r-hPDEl A and r-hPDElB respectively) which may be produced e.g., in HEK or SF9 cells by one skilled in the art. The PDEl enzyme is reconstituted with 50% glycerol to 2.5 U/ml. One unit of enzyme will hydrolyze 1.0 m of 3′,5′-cAMP to 5′-AMP per min at pH 7.5 at 30° C. One part enzyme is added to 1999 parts reaction buffer (30 μM CaCl₂, 10 U/ml of calmodulin (Sigma P2277), 10 mM Tris-HCl pH 7.2, 10 mM MgCl₂, 0.1% BSA, 0.05% NaN₃) to yield a final concentration of 1.25mU/ml. 99 l of diluted enzyme solution is added into each well in a flat bottom 96-well polystyrene plate to which 1 of test compound dissolved in 100% DMSO is added. The compounds are mixed and pre-incubated with the enzyme for 10 min at room temperature.

The FL-GMP conversion reaction is initiated by combining 4 parts enzyme and inhibitor mix with 1 part substrate solution (0.225 μM) in a 384-well microtiter plate. The reaction is incubated in dark at room temperature for 15 min. The reaction is halted by addition of 60 μL of binding reagent (1:400 dilution of IMAP beads in binding buffer supplemented with 1:1800 dilution of antifoam) to each well of the 384-well plate. The plate is incubated at room temperature for 1 hour to allow IMAP binding to proceed to completion, and then placed in an Envision multimode microplate reader (PerkinElmer, Shelton, CT) to measure the fluorescence polarization (Amp).

A decrease in GMP concentration, measured as decreased Amp, is indicative of inhibition of PDE activity. IC50 values are determined by measuring enzyme activity in the presence of 8 to 16 concentrations of compound ranging from 0.0037 nM to 80,000 nM and then plotting drug concentration versus AmP, which allows IC50 values to be estimated using nonlinear regression software (XLFit; IDBS, Cambridge, MA).

The Compounds of the Disclosure are tested in an assay as described or similarly described herein for PDE1 inhibitory activity. For example, Compound 1, is identified as a specific PDE1 inhibitor of formula:

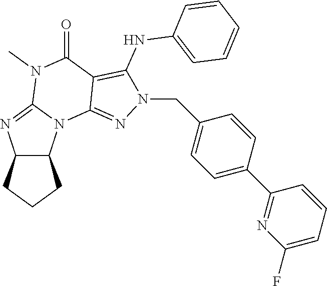 [SMILES: CN1C(=O)c2c(nn(Cc3ccc(-c4cccc(F)n4)cc3)c2Nc2ccccc2)N2C1=N[C@@H]1CCC[C@@H]12]

This compound has efficacy at sub-nanomolar levels vs PDE1 (IC₅₀ of 0.058 nM for bovine brain PDE1 in the assay described above) and high selectivity over other PDE families, as depicted on the following table:

| PDE Target | IC50 (nM) | ratio PDEx/PDE1* |
|---|---|---|
| hPDE1A | 0.027 | 1 |
| hPDE1B | 0.379 | 14 |
| hPDE1C | 0.037 | 1.4 |
| hPDE2A | 3661 | 135,593 |
| hPDE3B | 3120 | 115,556 |
| hPDE4D | 36 | 1,333 |
| r-bovine PDE5A | 632 | 23,407 |
| bovine retina PDE6 | 324 | 12,000 |
| hPDE7B | 355 | 13,148 |
| hPDE8A | 3001 | 111,148 |
| hPDE9A | 16569 | 613,666 |
| hPDE10A | 1824 | 67,666 |
| hPDE11A | 1313 | 48,629 |

The compound is also highly selective versus a panel of 63 receptors, enzymes, and ion channels. These data, and data for other PDE1 inhibitors described herein, are described in Li et al., J. Med. Chem. 2016: 59, 1149-1164, the contents of which are incorporated herein by reference.

### Example 2: Combination Therapy of PDE1 Inhibitor and Glucagon in Guinea Pig Cardiomyocytes

Studies were carried out to test the effect of glucagon co-administration with a PDE1 inhibitor in guinea pig cardiomyocytes. Myocytes were isolated according to Liu et al., Circulation Research, 2014. A thoracotomy was performed on anesthetized guinea pigs to remove the heart. The aorta was cannulated on a Langendorf apparatus fitted with a heating jacket circulating water at 37° C., and were retrogradely perfused for 5 minutes at 8 ml/min. The perfusate was switched to a Tyrode's solution containing collagen type 2 (Worthington) and protease type 14 (Sigma-Aldrich) for 7-9 minutes. The solution was switched to a modified Kraft-Bruhe (KB) buffer for 5 minutes, before being minced and filtered to yield single cells. Isolated cells were incubated in a modified KB buffer (Isenberg et al., Pflugers Arch, 1982] for an hour, before being placed in supplemented M199 ACCIT medium. (Ellingsen et al., American J Physiology, 1993) Cells were kept at room temperature, and used within 7 hours.

Changes in cell sarcomere shortening and Ca²⁺ transients were measured using a customized IonOptix system previously described. (Hashimoto et al., Circulation, 2018). Briefly, cells were loaded using 1 μM Fura-2 for 15 minutes, and washed out for at least 20 minutes. Baseline recording was made in Tyrode's buffer described above, with 0.1% DMSO. A first group of cells were then stimulated with glucagon (1 μM), and a second group of cells were stimulated with glucagon and Compound 1 (1 μM). Three baseline parameters were considered for the two types of measurements (diastolic value, peak percent change, and time to 50% baseline value). Only those cells falling within 2 standard deviations from the mean value were included for further analysis. All recordings were done at 37° C., with pacing at 1 Hz.

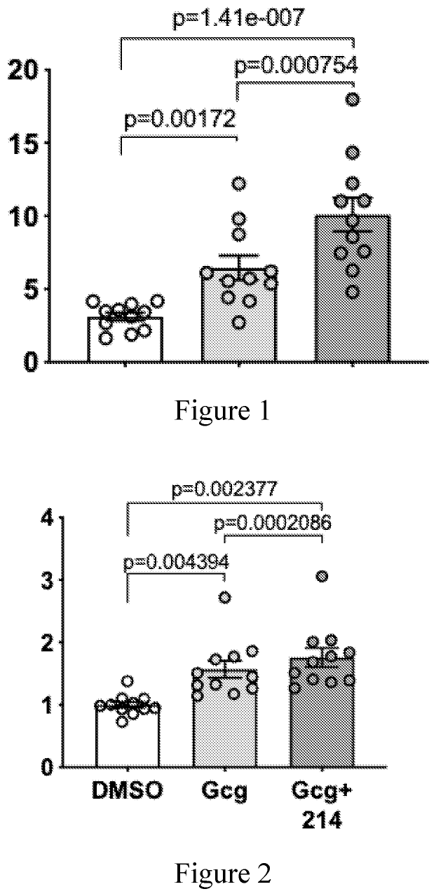

In this study cells were stimulated with glucagon alone, or with glucagon combined with Compound 1 as defined in Example 1. The grouped average change in cell sarcomere shortening are provided in FIG. 1, and the peak Ca2+ transient amplitude is demonstrated in FIG. 2. As shown, Compound 1 increased the beneficial effects of glucagon on both sarcomere shortening and peak Ca2+ transient amplitude.

Previously, the inventors have shown the PDE1 inhibition by Compound 1 in humans, dogs, rabbits and guinea pigs exerts beneficial effects on heart function by increasing cardiac index (cardiac power, output and contractility), and/or by decreasing systemic vascular resistance. The present results suggest that by combining a PDE1 inhibitor with agents that increase circulating glucagon, like SGLT2 inhibitors or glucagon alone, treatment of patients with heart failure may be synergistically improved (e.g. allowing lower doses to be used, yielding less adversities and improved outcomes).

1. A method for the treatment and/or prophylaxis of a disease, disorder or condition associated with altered glucagon function and/or with altered cyclic nucleotide signaling, comprising administration of a pharmaceutically effective amount of a PDE1 inhibitor and a pharmaceutically effective amount of an active agent that increases circulating glucagon to a patient in need thereof.

2. A method according to claim 1, wherein the condition, disease or disorder is associated with altered cyclic nucleotide signaling.

3. A method according to claim 1, wherein the condition, disease or disorder is associated with altered glucagon function.

4. A method according to claim 1, wherein the condition, disease or disorder is associated with altered cyclic nucleotide signaling and with altered glucagon function.

5. A method according to claim 1, wherein the condition, disease or disorder is selected from the group consisting of a cardiovascular condition, disease or disorder, a renal condition, disease or disorder, and a metabolic condition, disease or disorder.

6. A method according to claim 1, wherein the condition, disease or disorder is a cardiovascular condition, disease or disorder selected from the group consisting of angina, stroke, renal failure, essential hypertension, pulmonary hypertension, secondary hypertension, isolated systolic hypertension, hypertension associated with diabetes, hypertension associated with atherosclerosis, renovascular hypertension, congestive heart failure, an inflammatory disease or disorder, fibrosis, cardiac remodeling during gestation, cardiac hypertrophy, vascular remodeling, a connective tissue disease or disorder, chronic heart failure, acute heart failure, heart failure consequent to myocardial infarction, myocardial ischemia, myocardial hypoxia, reperfusion injury, left ventricular dysfunctions, myocardial infarction, ventricular expansion, vascular leakage, muscular dystrophy, and amyotrophic lateral sclerosis.

7. A method according to claim 1, wherein the condition, disease or disorder is a renal condition, disease or disorder selected from the group consisting of kidney fibrosis, chronic kidney disease, renal failure, glomerulosclerosis and nephritis, a renal disorder consequent to diabetes, an injury to a kidney, high blood pressure, a cancerous growth, and polycystic kidney disease.

8. A method according to claim 1, wherein the condition, disease or disorder is a metabolic disease, disorder or condition selected from the group consisting of type 1 diabetes mellitus, type 2 diabetes mellitus, high cholesterol, impaired glucose tolerance (IGT), impaired fasting blood glucose (IFG), hyperglycemia, postprandial hyperglycemia, overweight, obesity, metabolic syndrome, gestational diabetes, insulin resistance, conditions consequent to diabetes mellitus, conditions consequent to an abnormal accumulation of ectopic fat, hyperinsulemia, new onset diabetes after transplantation (NODAT), post-transplant metabolic syndrome (PTMS), focal segmented glomerulosclerosis, conditions consequent to NODAT and/or PTMS, hyperuricemia, kidney stones, hyponatremia, peripheral fluid retention and peripheral edema).

9. A method according to claim 1, wherein the PDE1 inhibitor is a compound selected from:
a. Formula I: 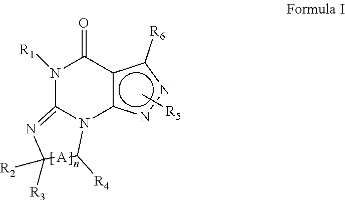
wherein
(i) R₁ is H or C₁₋₄ alkyl;
(ii) R₄ is H or C₁₋₄ alkyl and R₂ and R₃ are, independently, H or C₁₋₄ alkyl aryl, heteroaryl, arylalkoxy, heteroarylalkoxy, arylalkyl, or heteroarylaklyl; or
R₂ is H and R₃ and R₄ together form a di-, tri- or tetramethylene bridge preferably wherein the R₃ and R₄ together have the cis configuration;
(iii) R₅ is a substituted heteroarylalkyl;
or R₅ is attached to one of the nitrogens on the pyrazolo portion of Formula I and is a moiety of Formula A 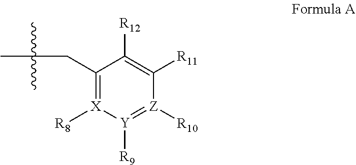
wherein X, Y and Z are, independently, N or C, and R₈, R₉, R₁₁ and R₁₂ are independently H or halogen, and R₁₀ is halogen, alkyl, cycloalkyl, haloalkyl, aryl, heteroaryl optionally substituted with halogen, thiadiazolyl, diazolyl, triazolyl, tetrazolyl, arylcarbonyl, alkylsulfonyl, heteroarylcarbonyl, or alkoxycarbonyl; provided that when X, Y, or Z is nitrogen, R₈, R₉, or R₁₀, respectively, is not present; and
(iv) R₆ is H, alkyl, aryl, heteroaryl, arylalkyl, arylamino, heteroarylamino, N,N-dialkylamino, N,N-diarylamino, or N-aryl-N-(arylalkyl)amino; and
(v) n=0 or 1;
(vi) when n=1, A is —C(R₁₃R₁₄)—
wherein R₁₃ and R₁₄, are, independently, H or C₁₋₄ alkyl, aryl, heteroaryl, arylalkoxy, heteroarylakoxy, arylalkyl, or heteroarylaklyl; in free, pharmaceutically acceptable salt or prodrug form, including its enantiomers, diastereoisomers and racemates;
b. Formula II: 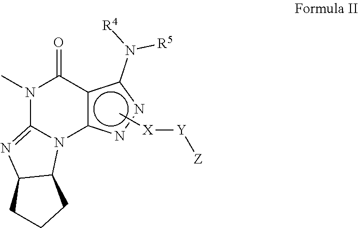
(i) X is C₁₋₆ alkylene;
(ii) Y is a single bond, alkynylene, arylene or heteroarylene;
(iii) Z is H, aryl, heteroaryl, halogen, haloC₁₋₆alkyl, —C(O)—R¹, —N(R²)(R³), or C₃₋₇ cycloalkyl optionally containing at least one atom selected from a group consisting of N or O;
(iv) R¹ is C₁₋₆alkyl, haloC₁₋₆alkyl, —OH or —OC₁₋₆ alkyl;
(v) R² and R³ are independently H or C₁₋₆ alkyl;
(vi) R₄ and R⁵ are independently H, C₁₋₆ alky or aryl optionally substituted with one or more halo, hydroxy or C₁₋₆ alkoxy;
(vii) wherein X, Y and Z are independently and optionally substituted with one or more halogen, C₁₋₆alkyl, haloC₁₋₆alkyl,
in free, pharmaceutically acceptable salt or prodrug form, including its enantiomers, diastereoisomers and racemates;
c. Formula III: 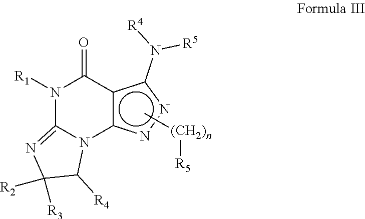
wherein
(i) R₁ is H or C₁₋₄ alkyl;
(ii) R₂ and R₃ are independently H or C₁₋₆ alkyl;
(iii) R₄ is H or C₁₋₄ alkyl;
(iv) R₅ is aryl optionally substituted with one or more groups independently selected from —C(═O)—C₁₋₆ alkyl and C₁₋₆-hydroxyalkyl;
(v) R₆ and R₇ are independently H or aryl optionally substituted with one or more groups independently selected from C₁₋₆ alkyl and halogen; and
(vi) n is 1, 2, 3, or 4,
in free, pharmaceutically acceptable salt or prodrug form, including its enantiomers, diastereoisomers and racemates;
d. Formula IV 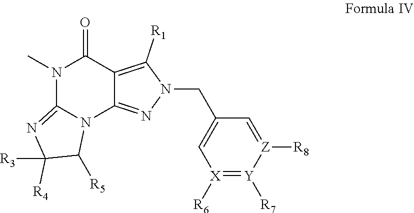
in free or salt form, wherein
(i) R₁ is C₁₋₄alkyl, or —NH(R₂), wherein R₂ is phenyl optionally substituted with halo;
(ii) X, Y and Z are, independently, N or C;
(iii) R₃, R₄ and R₅ are independently H or C₁₋₄alkyl; or R₃ is H and R₄ and R₅ together form a tri-methylene bridge preferably wherein the R₄ and R₅ together have the cis configuration,
(iv) R₆, R₇ and R₈ are independently:
H,
C₁₋₄alkyl,
pyrid-2-yl substituted with hydroxy, or
—S(O)₂—NH₂;
provided that when X, Y and/or Z are N, then R₆, R₇ and/or R₈, respectively, are not present; and when X, Y and Z are all C, then at least one of R₆, R₇ or R₈ is —S(O)₂—NH₂ or pyrid-2-yl substituted with hydroxy,
in free, pharmaceutically acceptable salt or prodrug form, including its enantiomers, diastereoisomers and racemates;
e. Formula 1a: 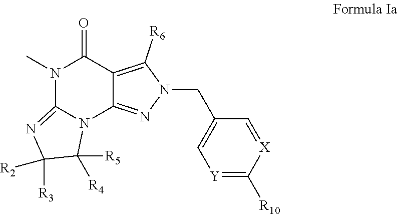
wherein
(i) R₂ and R₅ are independently H or hydroxy and R₃ and R₄ together form a tri- or tetra-methylene bridge preferably with the carbons carrying R₃ and R₄ having the R and S configuration respectively; or R₂ and R₃ are each methyl and R₄ and R₅ are each H; or R₂, R₄ and R₅ are H and R₃ is isopropyl preferably with the carbon carrying R₃ having the R configuration;
(ii) R₆ is phenylamino optionally substituted with halogen, benzylamino optionally substituted with halogen, C₁₋₄alkyl, or C₁₋₄ alkyl sulfide;
(iii) R₁₀ is C₁₋₄alkyl, methylcarbonyl, hydroxyethyl, carboxylic acid, sulfonamide, phenyl optionally substituted with halogen or hydroxy, pyridyl optionally substituted with halogen or hydroxy, or thiadiazolyl; and
X and Y are independently C or N,
in free, pharmaceutically acceptable salt or prodrug form, including its enantiomers, diastereoisomers and racemates;
f. Formula V 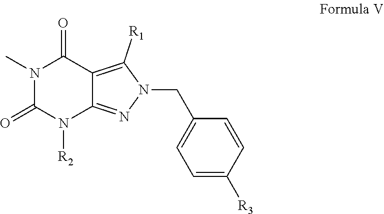
wherein
(i) R₁ is —NH(R₄), wherein R₄ is phenyl optionally substituted with halo;
(ii) R₂ is H or C₁₋₆alkyl;
(iii) R₃ is —SO₂NH₂ or —COOH;
in free, pharmaceutically acceptable salt or prodrug form, including its enantiomers, diastereoisomers and racemates; and/or
g. Formula VI 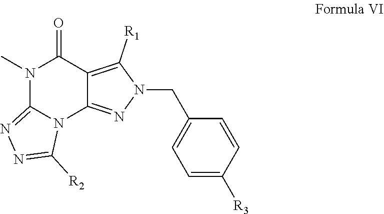
wherein
(i) R₁ is —NH(R₄), wherein R₄ is phenyl optionally substituted with halo;
(ii) R₂ is H or C₁₋₆alkyl;
(iii) R₃ is H, halogen, C₁₋₆alkyl, aryl optionally substituted with halogen, heteroaryl optionally substituted with halogen, or acyl,
in free, pharmaceutically acceptable salt or prodrug form, including its enantiomers, diastereoisomers and racemates;
h. Formula VII: 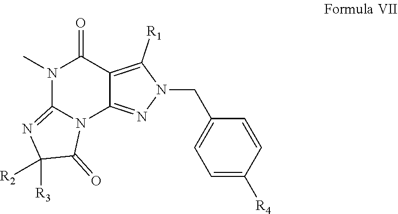
(i) R₁ is —NH(R₅), wherein R₅ is phenyl optionally substituted with halo;
(ii) R₂ and R₃ are each independently H or C₁₋₆alkyl;
(iii) R₄ is aryl optionally substituted with halogen or heteroaryl optionally substituted with halogen,
in free, pharmaceutically acceptable salt or prodrug form, including its enantiomers, diastereoisomers and racemates.

10. A method according to claim 1, wherein the PDE1 inhibitor is selected from any of the following: 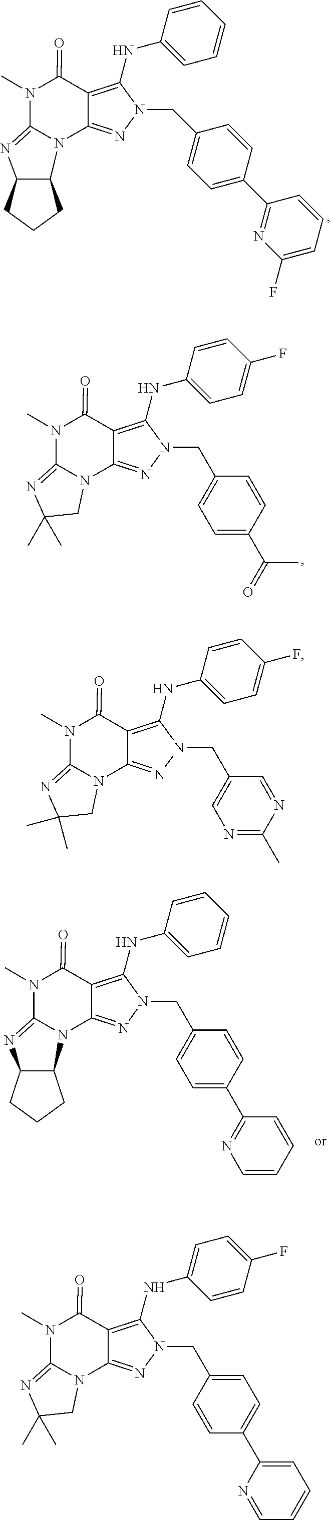
in free or pharmaceutically acceptable salt form.

11. A method according to claim 1, wherein the active agent that increases circulating glucagon is selected from glucagon, atigliflozin, canagliflozin, dapagliflozin, empagliflozin, ertugliflozin, ipragliflozin, luseogliflozin, remogliflozin, sergliflozin, sotagliflozin, tofogliflozin, and phlorizin, in free or pharmaceutically acceptable salt form.

12. A method according to claim 1, wherein the active agent that increases circulating glucagon is selected from glucagon, canagliflozin, dapagliflozin, empagliflozin, ertugliflozin, in free or pharmaceutically acceptable salt form.

13. A method according to claim 1, wherein the active agent that increases circulating glucagon is dapagliflozin, in free or pharmaceutically acceptable salt form.

14. A method according to claim 1, wherein the active agent that increases circulating glucagon is dapagliflozin, in free form.

15. A method according to claim 1, further comprising administering to the patient a pharmaceutically effective amount of a SGLT2 inhibitor.

16. The method of claim 8, wherein said condition consequent to diabetes mellitus is selected from the group consisting of cataracts and micro- and macrovascular diseases, such as nephropathy, retinopathy, neuropathy, tissue ischemia, arteriosclerosis, myocardial infarction, stroke and peripheral arterial occlusive disease, and degeneration or decline in the functionality of pancreatic beta cells.
